(19) United States
(12) Patent Application Publication
(10) Pub. No.: US 2022/0267799 A1
(43) Pub. Date: Aug. 25, 2022

(54) GENETICALLY MODIFIED ENTEROVIRUS VECTORS

(71) Applicant: VIROGIN BIOTECH CANADA LTD, Richmond (CA)

(72) Inventors: Honglin Luo, Vancouver (CA); William Wei-Guo Jia, Vancouver (CA); Huitao Liu, Vancouver (CA); Ismael Samudio, Vancouver (CA)

(21) Appl. No.: 17/633,181

(22) PCT Filed: Aug. 5, 2020
(86) PCT No.: PCT/US20/45069

§ 371 Date: Feb. 4, 2022

Related U.S. Application Data
(60) Provisional application No. 62/883,055, filed on Aug. 5, 2019.

(51) Int. Cl. C12N 15/86; A61K 35/768; C12N 7/00; A61P 35/00

(52) U.S. Cl. CPC C12N 15/86; A61K 35/768; C12N 7/00; A61P 35/00; C12N 2770/32321; C12N 2770/32332; C12N 2770/32343

(57) ABSTRACT

A replicating oncolytic virus vector is provided having a modified Enterovirus genome (e.g., a Poliovirus, Coxsackievirus or Echovirus genome), wherein the modified Enterovirus genome has one or more copies of one or more miRNA target sequences operably linked to an untranslated region (UTR) of the Enterovirus genome. Also provided are compositions and methods for treating cancer (including for example, lung cancer).

### CROSS-REFERENCE TO RELATED APPLICATION

This patent application claims the benefit under 35 U.S.C. § 119(e) of U.S. Provisional Patent Application No. 62/883,055 filed Aug. 5, 2019, which application is incorporated herein by reference in its entirety for all purposes.

### FIELD OF THE INVENTION

The present invention relates generally to genetically modified oncolytic Enterovirus vectors, and uses thereof which have reduced toxicity in normal tissues.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “VIRO412_ST25.txt”, a creation date of Aug. 4, 2019, and a size of 2.20 KB. The Sequence Listing filed via EFS-Web is part of the specification and is incorporated in its entirety by reference herein.

### BACKGROUND

Cancer includes a wide variety of diseases that involve the uncontrolled or abnormal growth of cells that spread or invade into other tissues of the body, initially resulting in changes in bodily function (depending on the type of cancer), and ultimately in death. About 14.1 million new cases of cancer occur each year (excluding skin cancer other than melanoma).

In much of the Western world, lung cancer is the 3rd and 2nd most common cancer for men and women, respectively, and the leading cause of cancer-related deaths for both sexes. Non-small-cell lung cancer (“NSCLC”) constitutes ˜85% of lung cancer cases. Among them, adenocarcinoma is the most common type of lung cancer, accounting for almost half of all lung cancers. Genetic mutations play critical roles in the development of NSCLC. Well-identified oncogenic driver mutations include epidermal growth factor receptor (“EGFR”) and Kirsten rat sarcoma viral oncogene homolog (“KRAS”), which occur in ˜15% and ˜30% of lung adenocarcinoma, respectively Although EGFR mutations can be clinically targeted, KRAS mutations are currently very difficult to treat and associated with a poor prognosis. Small-cell lung cancer (“SCLC”) accounts for ˜15% of all lung cancers. Between 60% to 90% of SCLC cases are featured by mutations in gene encoding tumor protein p53 and/or retinoblastoma protein (Rb). There is also no targeted therapy for SCLC.

A number of therapies have been developed to treat cancer, including for example, radiation therapy, chemotherapy, surgical removal of the cancer, or some combination of these therapies. One new area of therapy that has shown progress is ‘targeted therapy’, wherein compositions and methods are used to specifically target and kill tumor cells (as opposed to ‘normal’ cells).

One example of a targeted therapy are oncolytic viruses. Briefly, an oncolytic virus is defined as one that is capable of inducing lysis of tumor cells via its self-replication process, and preferably, without causing substantive damage to normal tissues. The greatest advantage of oncolytic viruses over other cancer therapies is that the candidate viruses can be genetically manipulated to increase their potency against specific cancer types. In 2015, the FDA approved the first genetically modified herpes simplex virus 1 (Talimogene laherparepvec or “T-VEC”) for the treatment of melanoma. Over the past decades, several different oncolytic viruses, including retrovirus, vaccinia virus, adenovirus, measles virus, and Newcastle disease virus, have all been tested in clinical trials for the treatment of cancer. However, the overall anti-cancer efficacy and specificity remain low and there is still no FDA-approved virotherapy for lung cancer.

Hence, there remains a need for improved targeted treatments for cancer, e.g., lung cancer, that lyse and destroy transformed cells, while not causing substantive damage to healthy, untransformed cells, and which overcome one or more of the shortcomings associated with the prior art.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to micro-RNA (“miRNA”) based approaches to modify an Enterovirus genome (e.g., a Coxsackievirus such as B3) in order to further enhance its tumor-specificity.

In one aspect, the invention provides a replicating oncolytic virus vector (i.e., a recombinant vector) having a modified Enterovirus genome (e.g., a Poliovirus, Coxsackievirus or Echovirus genome), wherein the modified Enterovirus genome (e.g., a Poliovirus, Coxsackievirus or Echovirus genome) includes one or more copies of one or more miRNA target sequences. Within preferred embodiments the miRNA target sequences are operably linked to an untranslated region (UTR) of the Enterovirus genome (e.g., a Poliovirus, Coxsackievirus or Echovirus genome). In some embodiments, the Coxsackievirus is Coxsackievirus A or Coxsackievirus B. Within certain embodiments the Enterovirus is Coxsackievirus B3. In other embodiments, the untranslated region (UTR) is a 5′ UTR, and/or a 3′UTR.

In some embodiments, the one or more copies of the one or more_miRNA target sequences include one or more copies of two or more different miRNA target sequences. In other embodiments, the two or more different miRNA target sequences recognize an miRNA selected the group consisting of miR-1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR-216, miR-217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*. In other embodiments, the two or more different miRNA target sequences include target sequences for miR-145 and miR-143. In yet other embodiments, the two or more different miRNA target sequences include four copies of the target sequence for miR-145 and two copies of the target sequence for miR-143. In other embodiments, one or more copies of the one or more miRNA target sequences is in a forward orientation and/or one or more copies of the one or more miRNA target sequences is in a reverse orientation. In some embodiments, the recombinant vector further includes at least one nucleic acid encoding a non-viral protein selected from the group consisting of immunostimulatory factors, antibodies, and checkpoint blocking peptides, wherein the at least one nucleic acid is operably linked to a suitable tumor-specific regulatory region. In other embodiments, the non-viral protein is selected from the group consisting of IL12, IL15, IL15 receptor alpha subunit, OX40L, and a PD-L1 blocker.

In another aspect, the invention provides a method for lysing tumor cells, comprising providing an effective amount of a replicating oncolytic virus vector of any of the above embodiments to tumor cells. In some embodiments, the tumor cells include lung cancer cells. Inn other embodiments the tumor cells include pancreatic cells.

In other aspects, the invention provides a therapeutic composition including at least one replicating oncolytic virus vector of any of the above embodiments and a pharmaceutically acceptable carrier.

In other aspects, the invention provides a method for treating cancer in a subject suffering therefrom, including the step of administering a first composition comprising a therapeutically effective amount of the composition of any of the above embodiments. In some embodiments, the cancer is non-small-cell lung cancer (NSCLC) or small-cell lung cancer (SCLC). In other embodiments, the administration is intravenous (IV) administration, intraperitoneal (IP) administration, or intratumoral (IT) administration.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIGS. 1A and 1B are histogram graphs showing relative expression levels of miRNAs.

FIG. 2 is a schematic illustration depicting construction one embodiment of a modified oncolytic Coxsackievirus B3 (“CVB3”) genome.

FIGS. 3A, 3B and 3C are graphs showing viral titers and RNA copies in cell lines infected with oncolytic CVB3 viruses.

FIGS. 4A, 4B, 4C and 4D are photographs and graphs depicting data from cell lines infected with oncolytic CVB3 viruses.

FIGS. 5A, 5B and 5C are photographs and graphs depicting data from cell lines infected with oncolytic CVB3 viruses.

FIGS. 6A, 6B, 6C and 6D are photographs and graphs depicting data from cell lines infected with oncolytic CVB3 viruses.

FIGS. 7A, 7B, 7C, 7D, 7E and 7F are survival rate plots, histological photographs, and graphs depicting data from mouse model systems (SCID mice) treated with oncolytic CVB3 viruses.

FIGS. 8A, 8B, 8C, 8D, 8E, 8F and 8G are survival rate plots, histological photographs, and graphs depicting data from mouse model systems treated with oncolytic CVB3 viruses.

FIGS. 9A, 9B, 9C and 9D are a schematic illustration depicting construction of three additional miRNA-modified CVB3, and body weight changes, survival rates, and histological photographs from mouse model systems (C57BL/6 mice) treated with these oncolytic CVB3 viruses.

FIGS. 10A-10Z, 10AA-10ZZ, and 10AAA-10SSS are a selected list of microRNAs in tumors, all of which are incorporated by reference in their entirety.

FIGS. 11A and 11B are photographs and cell viability plots depicting data from cell lines infected with oncolytic CVB3 viruses.

FIGS. 12A, 12B, 12C, 12D, 12E, and 12F are histological photographs, graphs depicting data, schematic illustrations of oncolytic CVB3 viruses, and survival rate plots from an immunocompetent mouse model system treated with these novel oncolytic CVB3 viruses.

FIGS. 13A and 13B are histological photographs and graph depicting viral RNA copy numbers from C57BL/6 mice injected with various oncolytic CVB3 viruses.

FIGS. 14A and 14B are graphs depicting cell viability and photographs of cell lines infected with various oncolytic CVB3 viruses.

FIGS. 15A, 15B, 15C, and 15D are descriptions of various cancer cell lines, photographs of these cell lines infected with various oncolytic CVB3 viruses, schematic illustrations of new oncolytic CVB3 viruses, and photographs of cell lines infected with these new oncolytic CVB3 viruses.

### DETAILED DESCRIPTION OF THE INVENTION

The present invention may be understood more readily by reference to the following detailed description of preferred embodiments of the invention and the Examples included herein.

Prior to setting forth the invention in more detail however, it may be helpful to an understanding thereof to first set forth definitions of certain terms that are used hereinafter.

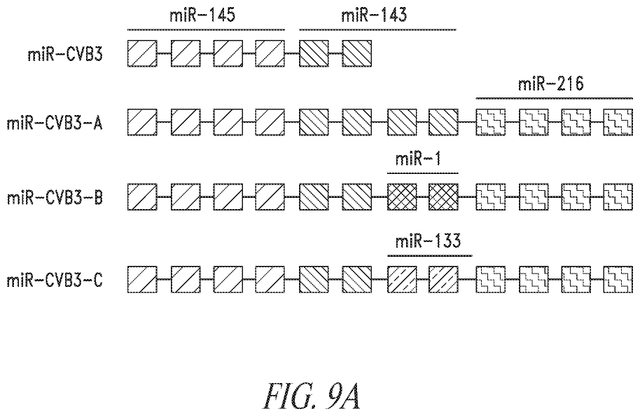

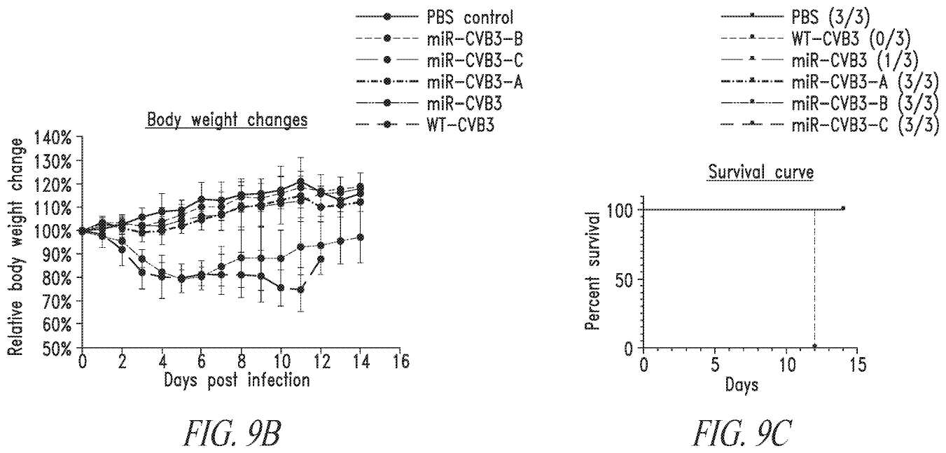

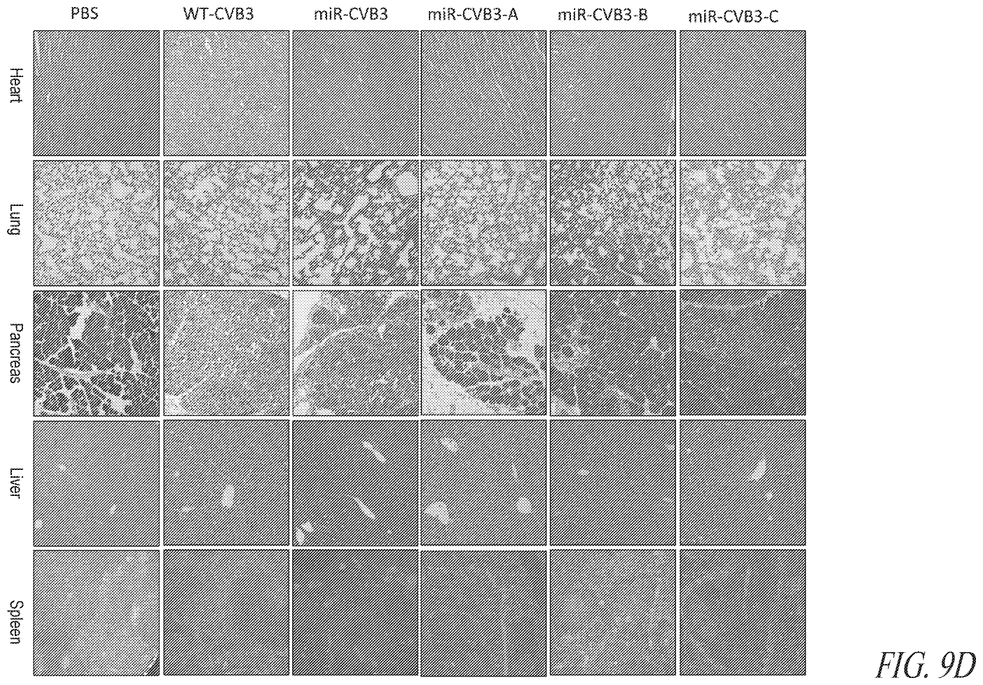

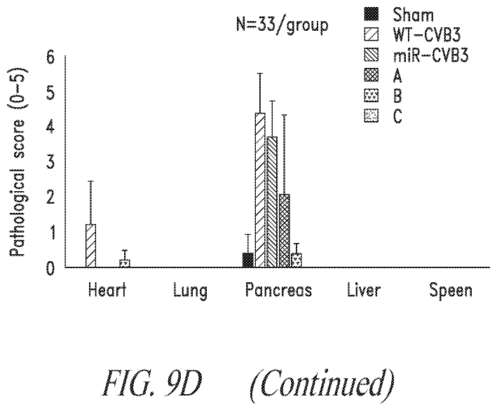

The term “microRNA” or “miRNA” as used herein refers to a family of short (typically 21-25 nucleotides), endogenous, single-stranded RNAs expressed in a wide range of organisms including both animals and plants. There are over 1000 unique miRNAs expressed in humans. miRNAs bind to specific target sequences found in messenger RNAs (mRNAs). Binding to complementary or partially complementary sequences (target sequences) in mRNA molecules results in down-regulation of gene expressing by cleavage of the mRNA, increased degradation from shortening of its polyA tail, and direct translational repression. A selected list of microRNAs in tumors (along with associated references) are provided in FIGS. 9A-9Z, 9AA-9ZZ, and 9AAA-9SSS, which list and associated references are incorporated by reference in their entirety.

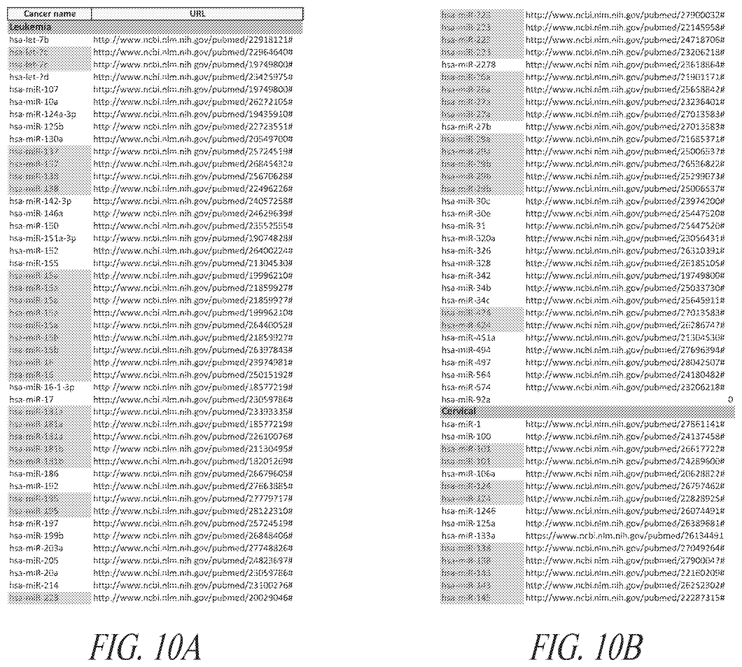

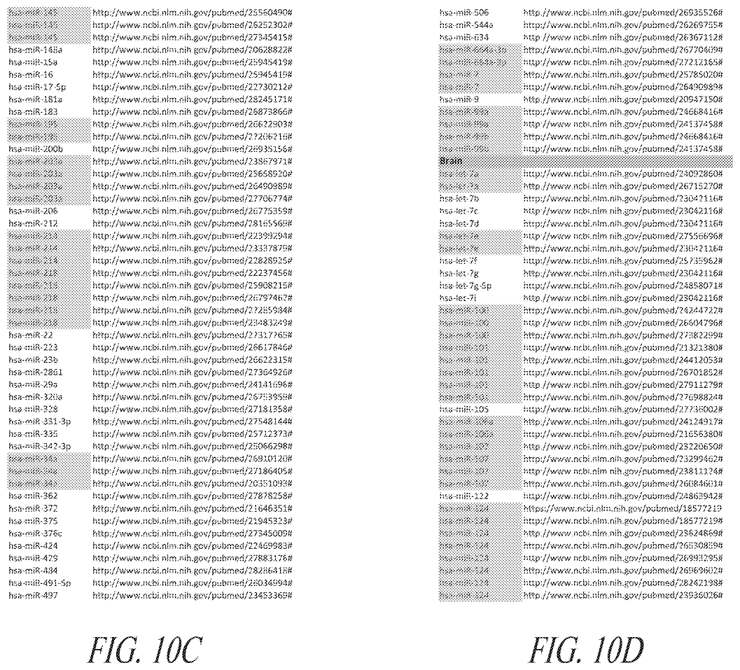

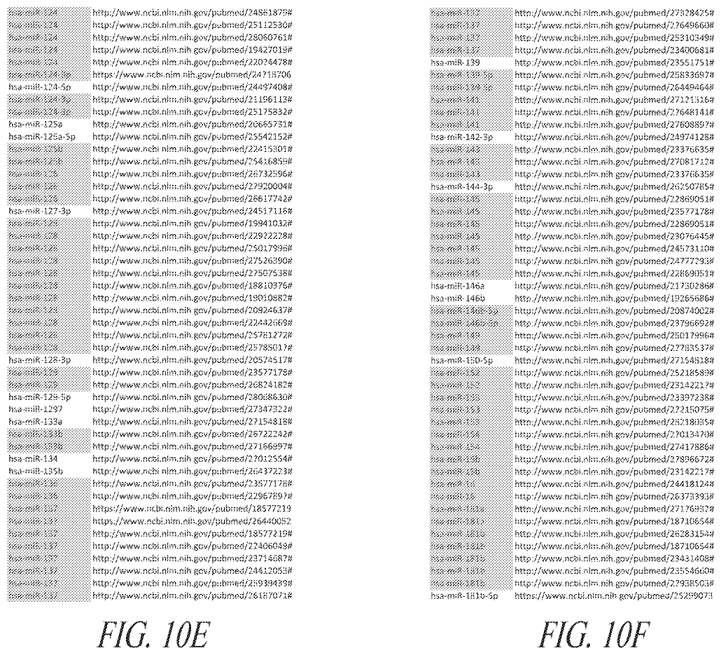

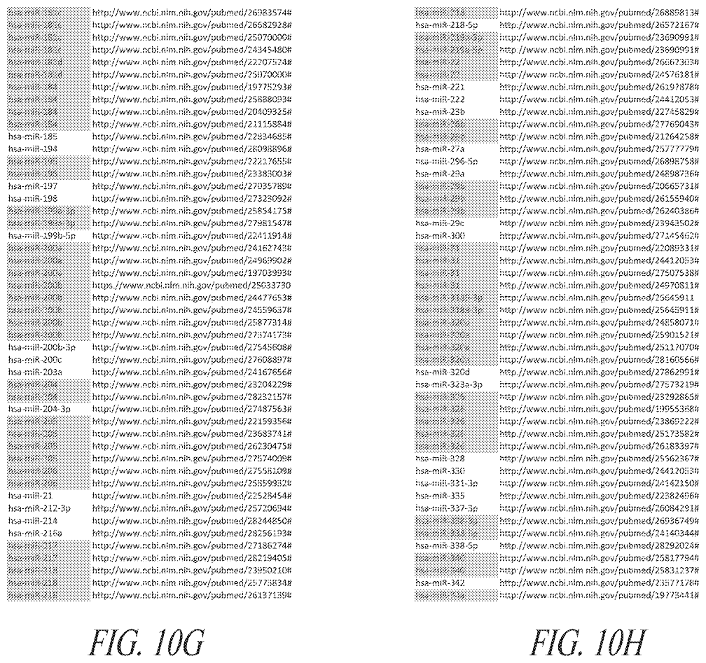

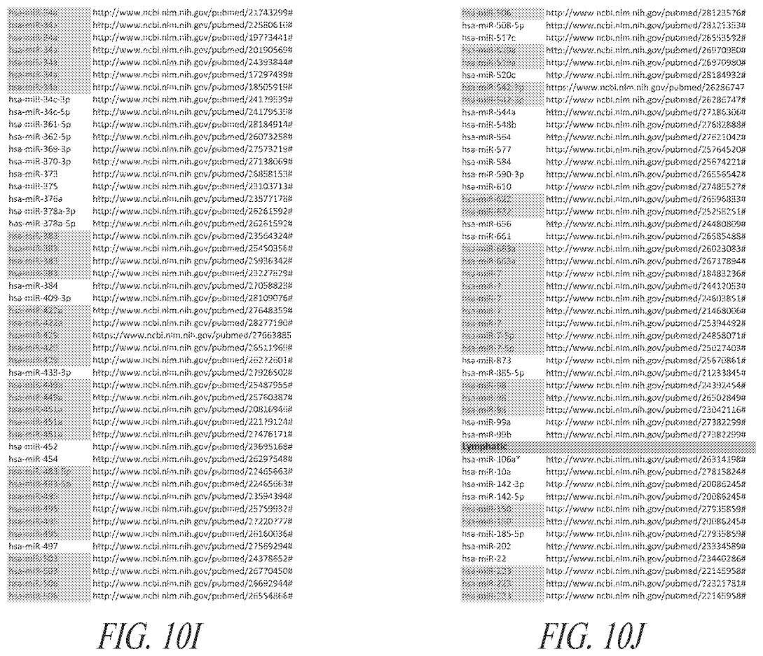

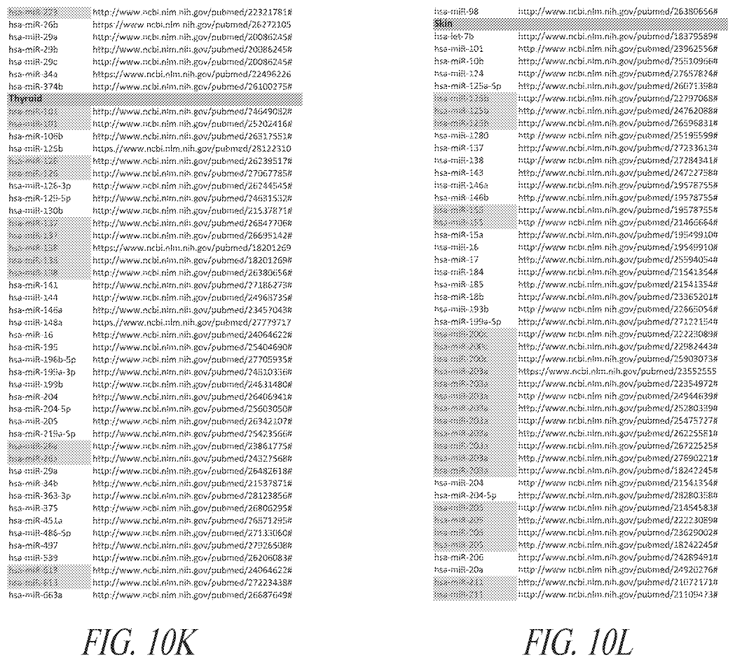

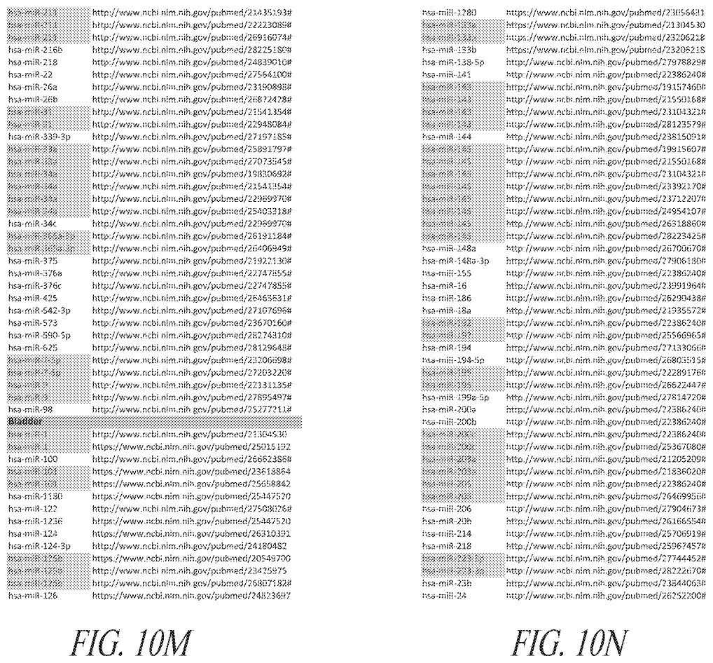

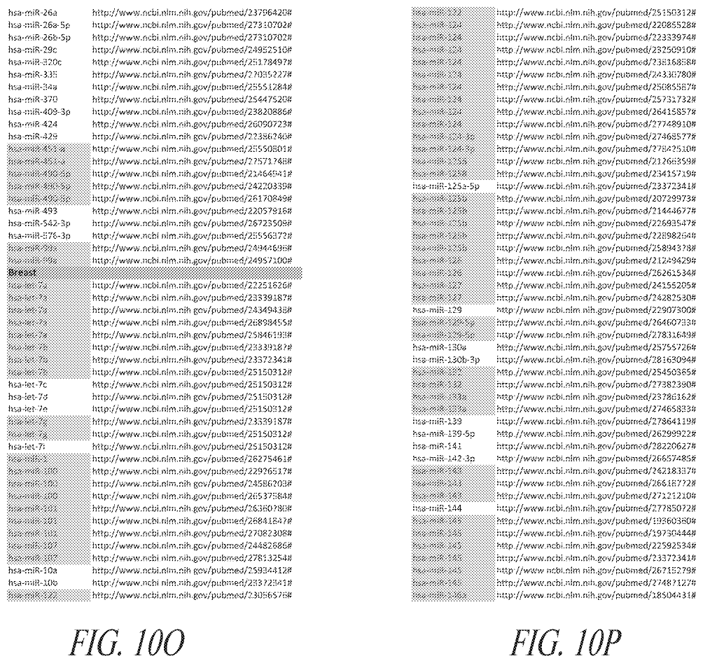

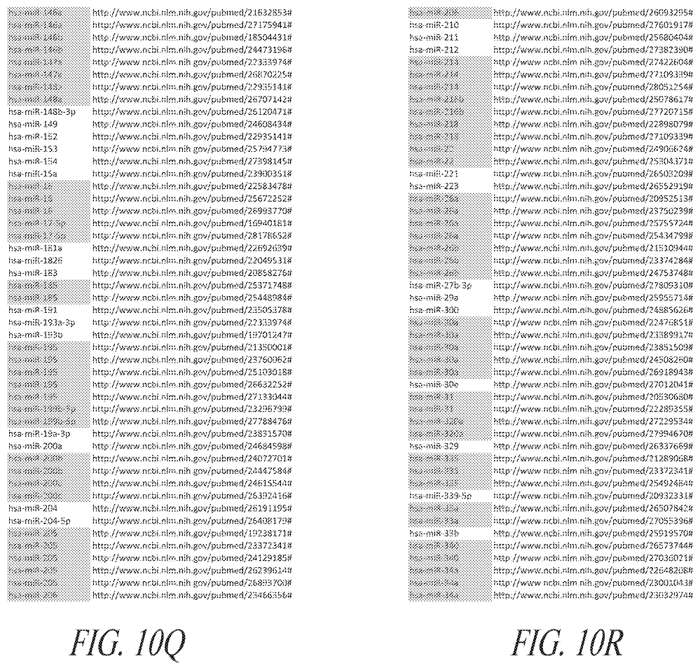

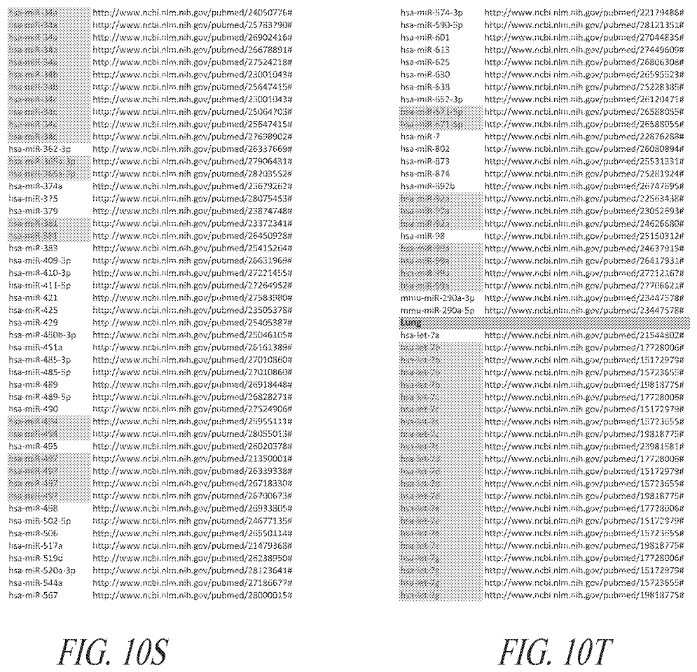

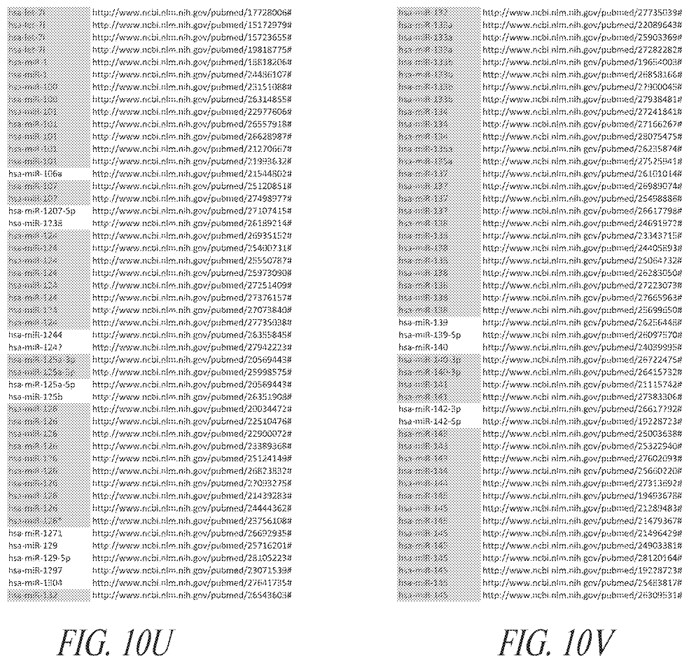

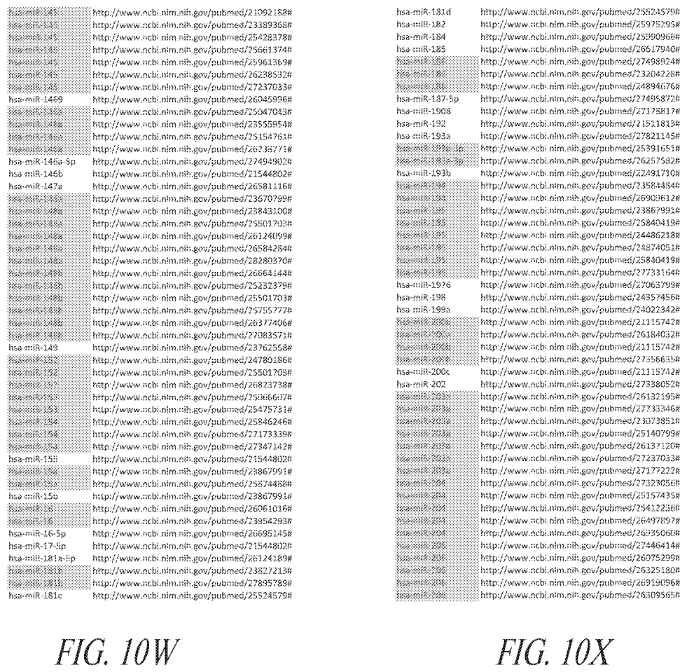

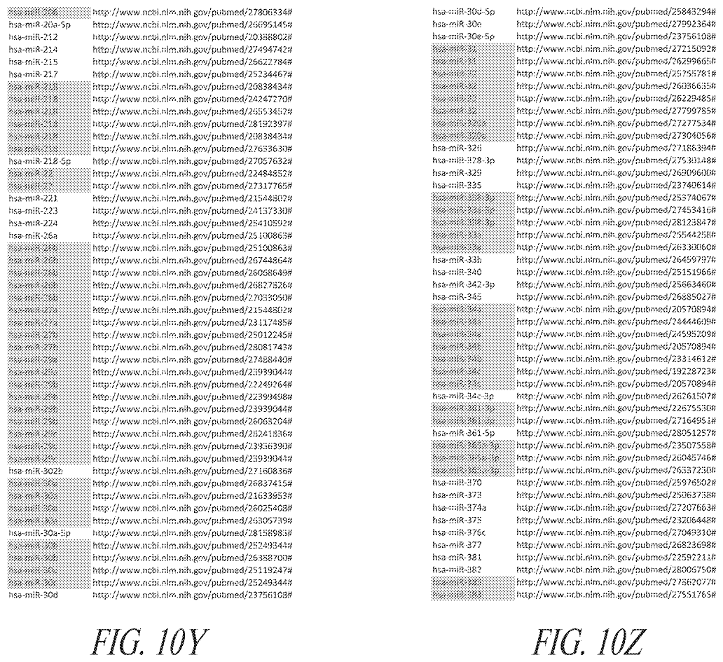

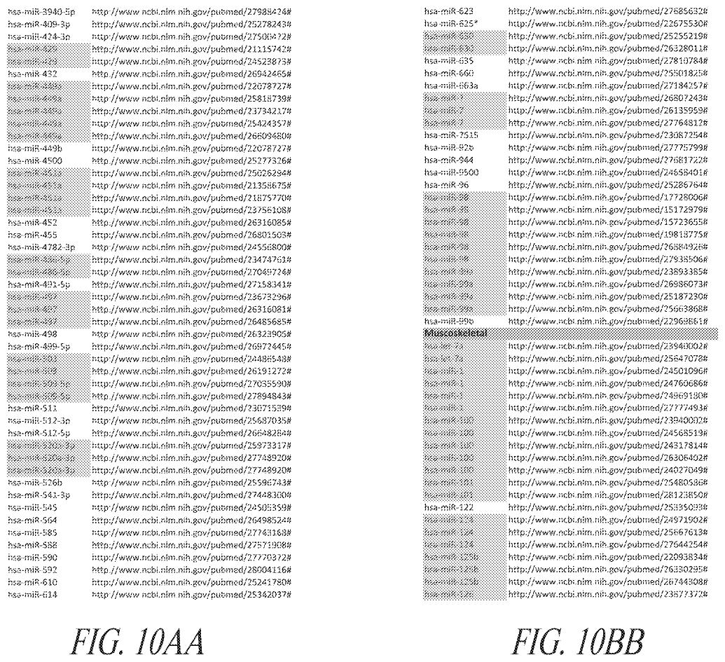

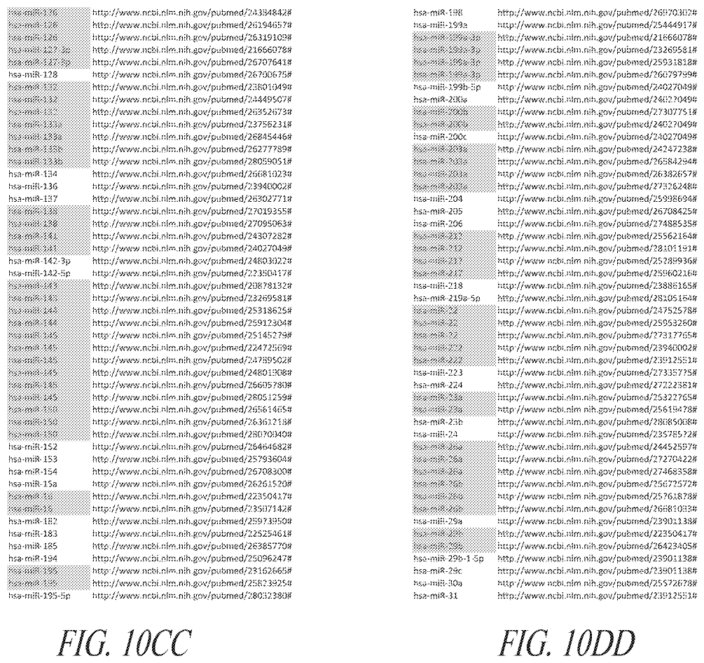

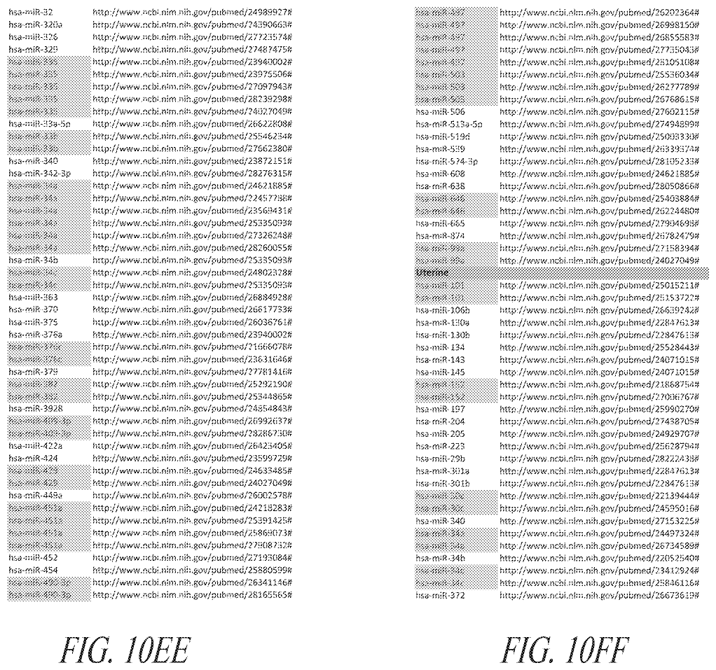

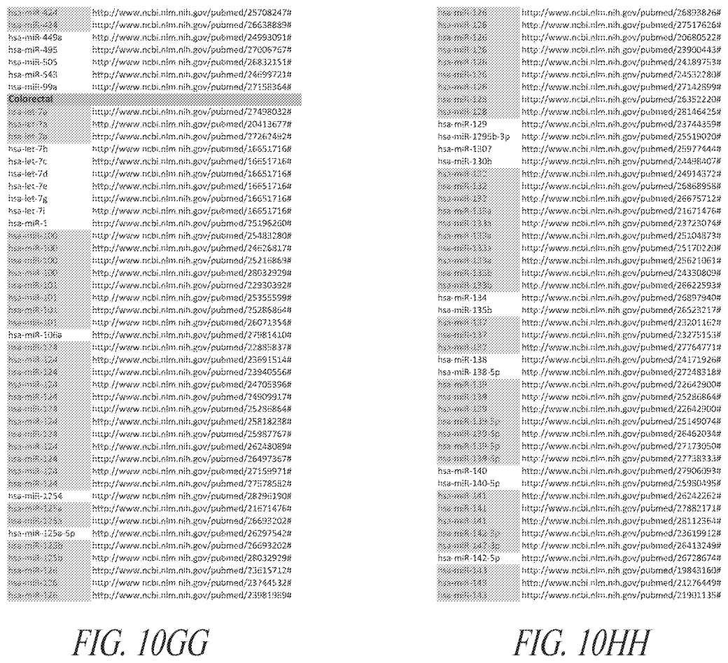

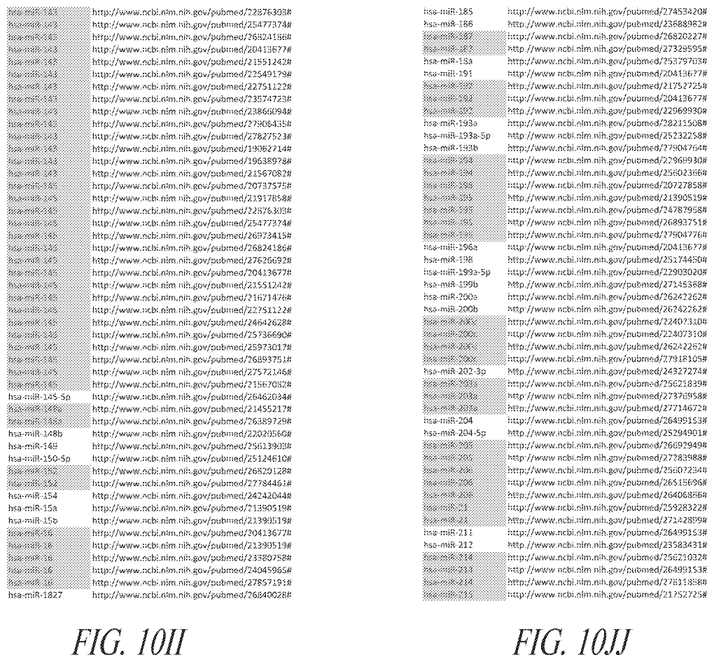

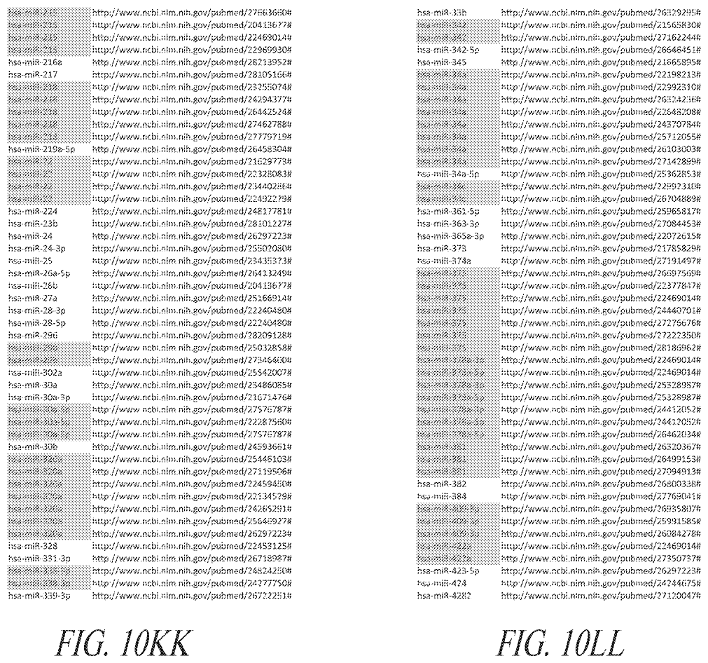

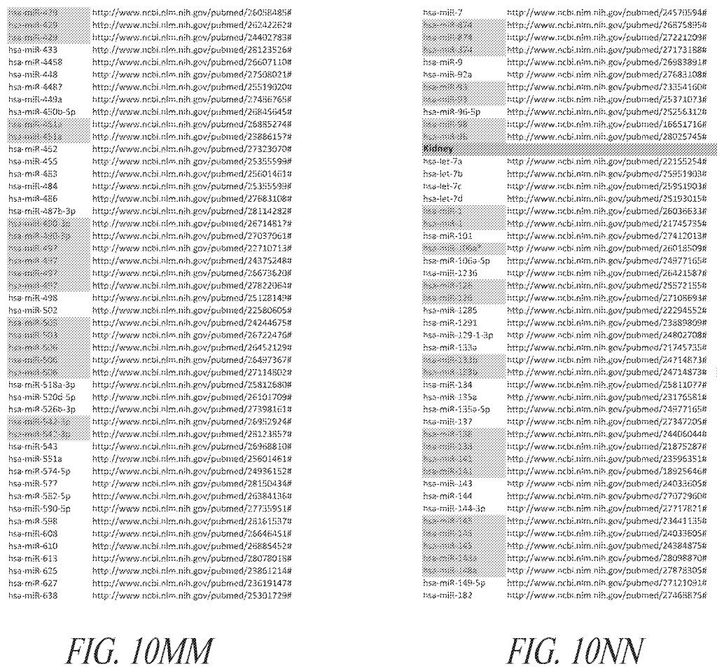

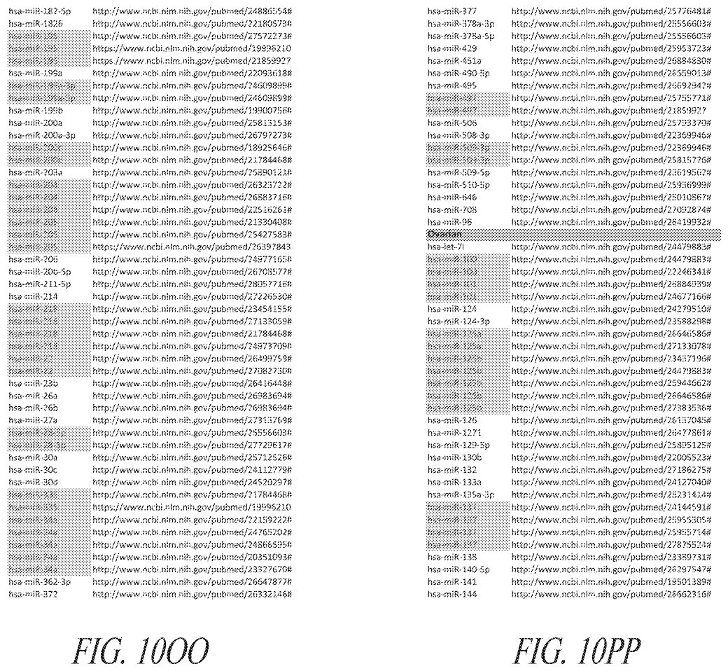

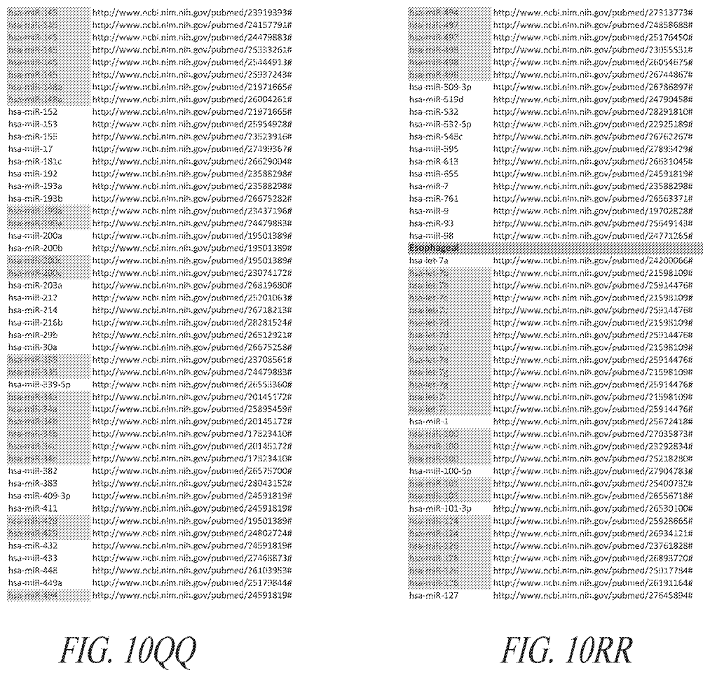

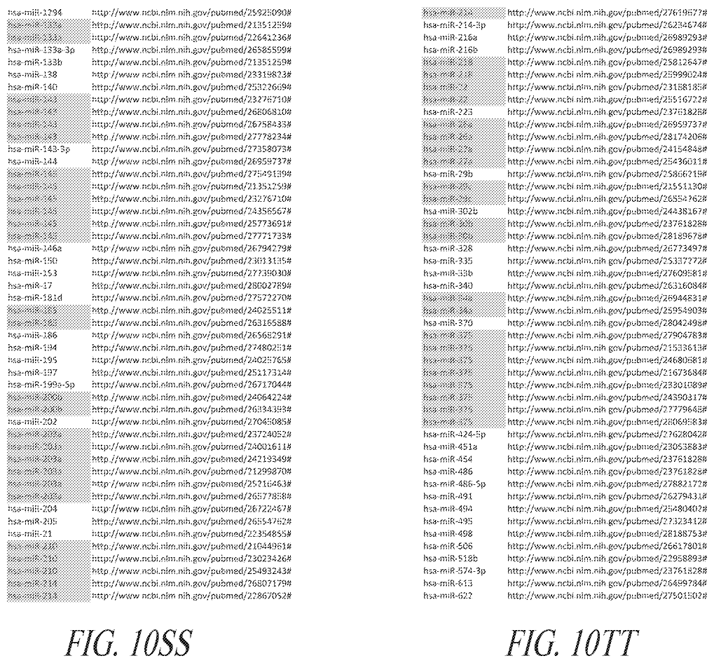

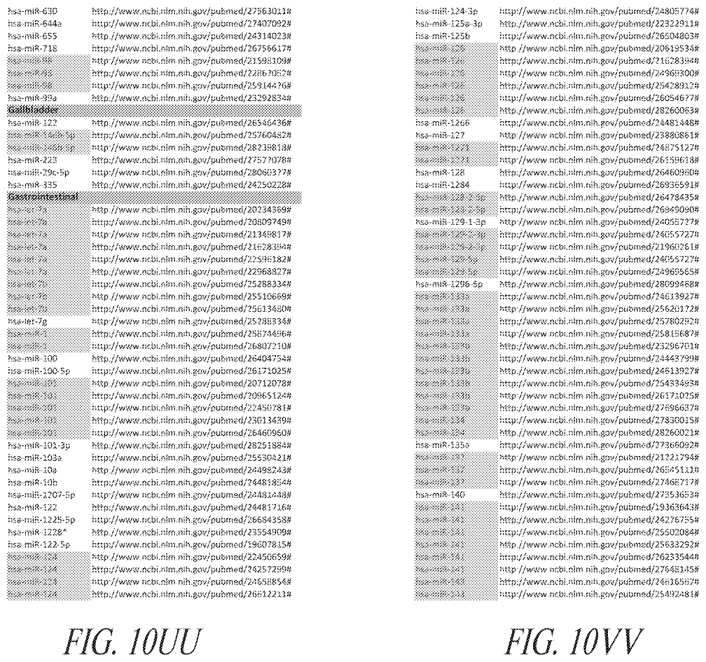

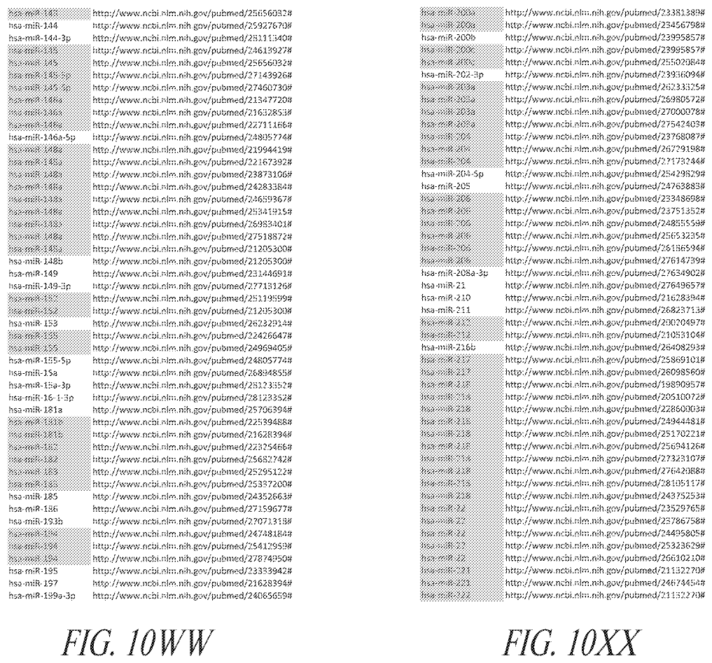

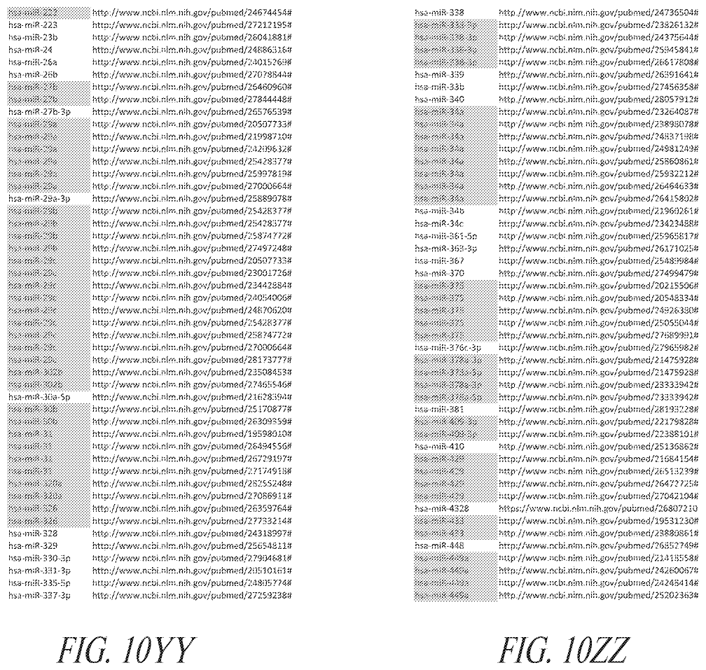

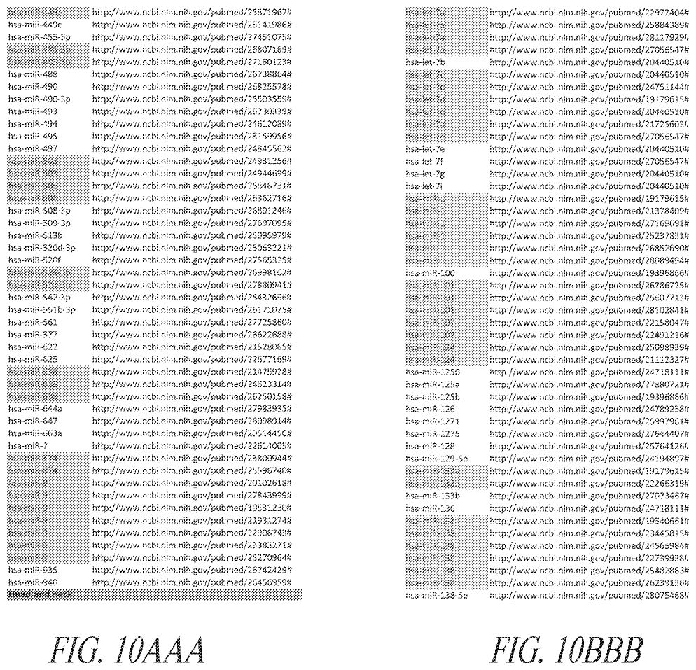

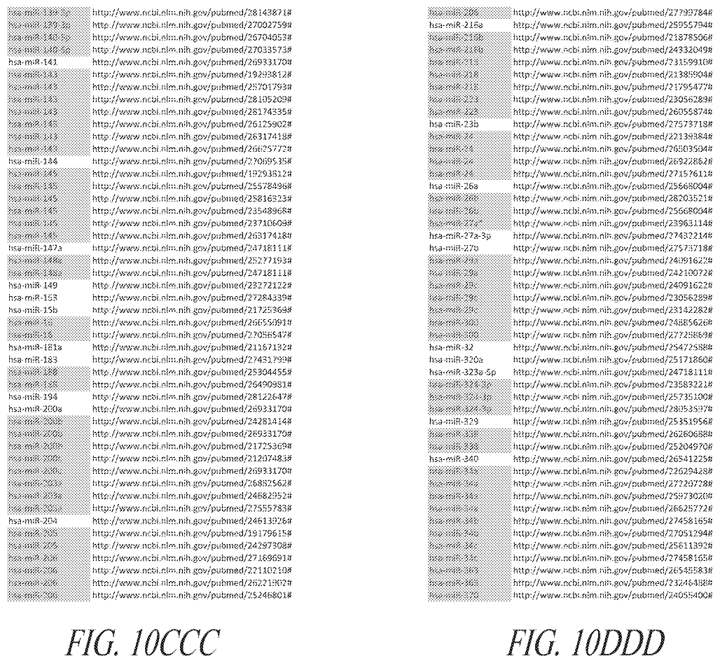

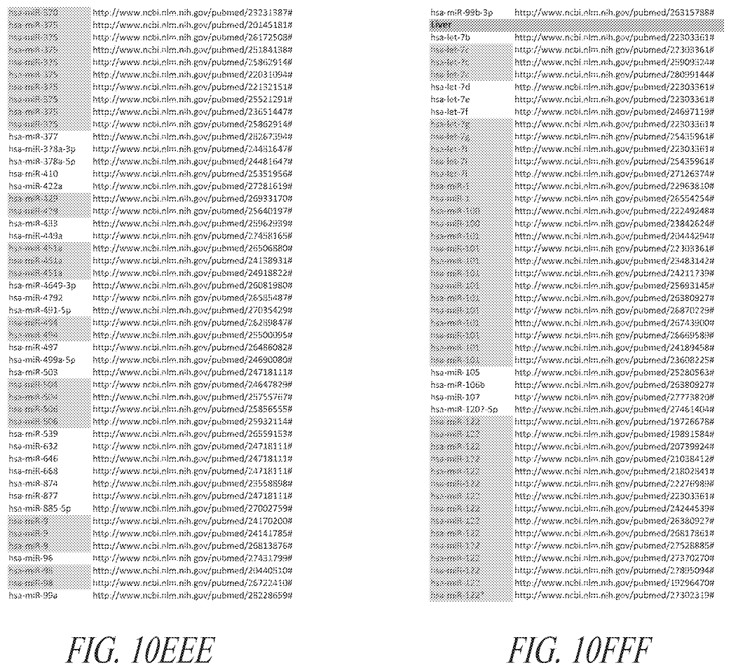

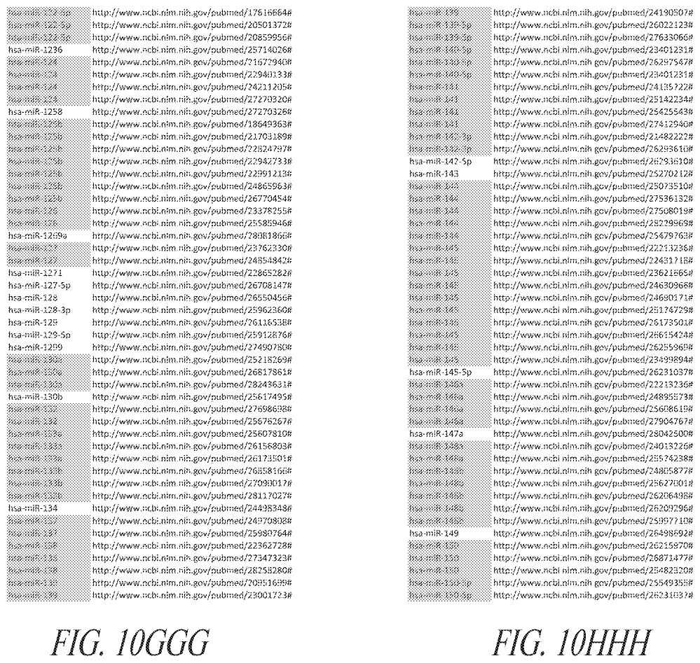

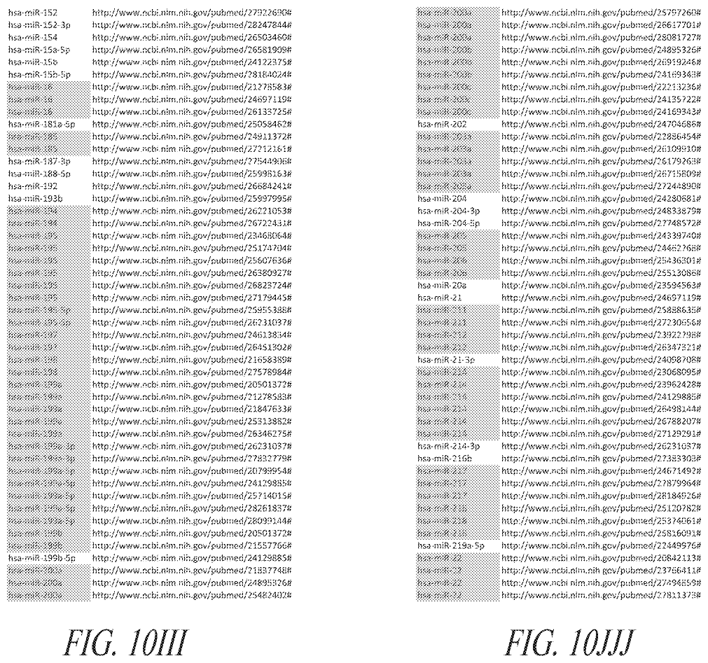

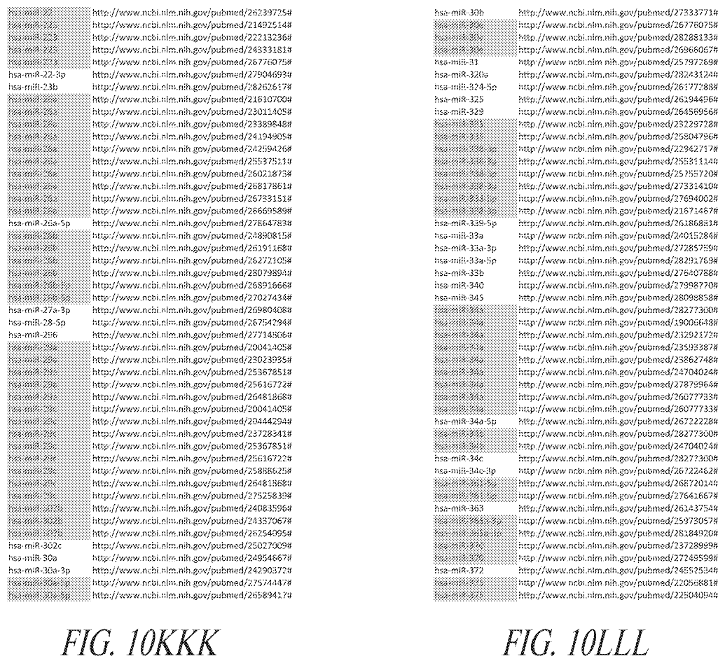

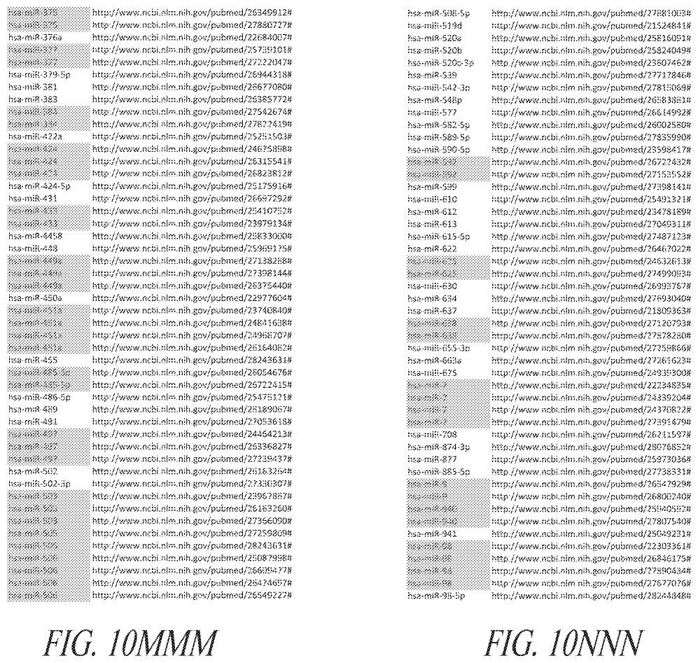

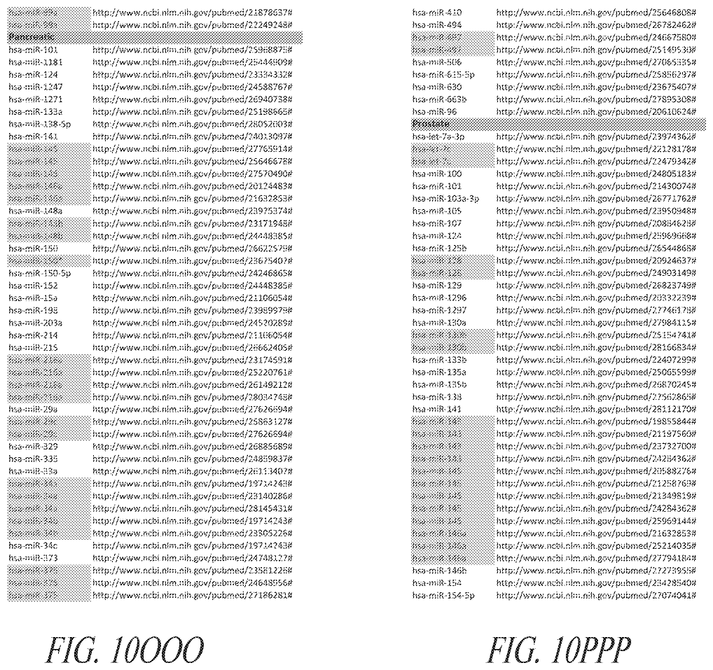

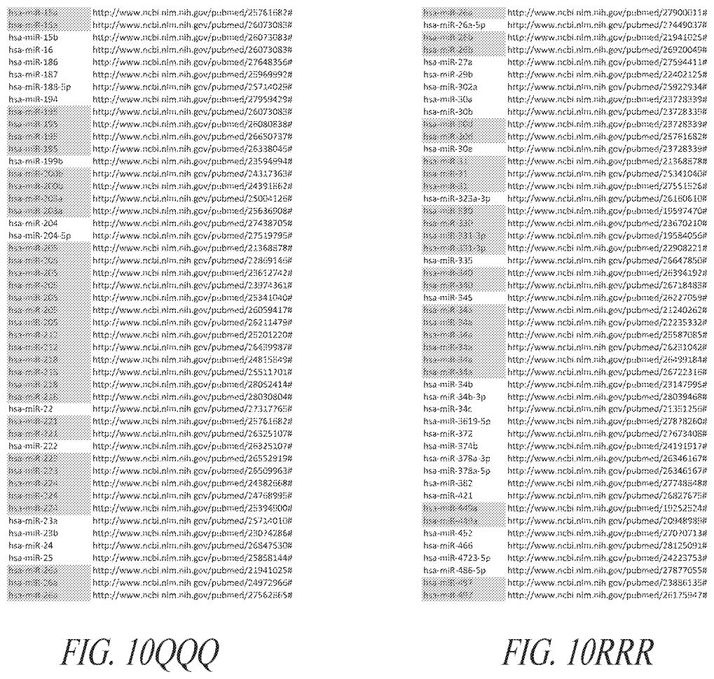

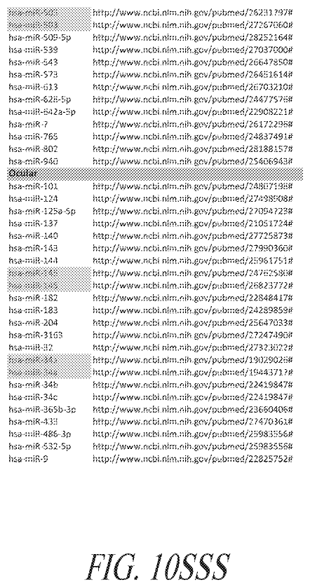

“MicroRNA target sequence(s)”, “miRNA target sequence(s)” and “miRNA binding sequence(s)” refer to sequences which are complementary to, or bind to (i.e., they need not be 100% complementary) to miRNA sequences such as those disclosed in FIG. 10.

The term “oncolytic Enterovirus” refers to a Enterovirus that is capable of replicating in and killing tumor cells. Briefly, Enterovirus is a genus of single stranded positive-sense RNA viruses which are most commonly associated with mammalian diseases that are transmitted through a fecal-oral route. Common examples of Enterovirus include poliovirus, coxsackievirus and echoviruses.

The term “oncolytic Coxsackievirus” or “CSV” refers generally to a Coxsackievirus capable of replicating in and killing tumor cells. Within certain embodiments the virus can be recombinantly (or ‘genetically’) engineered in order to more selectively target tumor cells and/or to reduce immune-mediated neutralization of the CSV in a human host. Coxsackievirus B3 (CVB3) is a small, nonenveloped virus that contains a positive RNA genome encoding a single open reading frame flanked by 5′ and 3′ untranslated regions (UTRs).

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

The term “cancer” refers to a disease state caused by uncontrolled or abnormal growth of cells in a subject. Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Benign tumors and other conditions of unwanted cell proliferation may also be treated.

In order to further an understanding of the various embodiments herein, the following sections are provided which describe various embodiments: A. Oncolytic Enteroviruses; B. MicroRNAs; D. Therapeutic Compositions, and E. Administration.

A. Oncolytic Enteroviruses

As noted above, Enteroviruses are a genus of single stranded positive-sense RNA viruses which are most commonly associated with mammalian diseases that are transmitted through a fecal-oral route. Common examples of Enterovirus include polioviruses, coxsackieviruses and echoviruses.

Coxsackievirus is a virus that belongs to a family of nonenveloped, linear, positive-sense single-stranded RNA viruses, Picornaviridae and the genus Enterovirus, which also includes poliovirus and echovirus. Enteroviruses are among the most common and important human pathogens, and ordinarily its members are transmitted by the fecal-oral route. Coxsackieviruses are among the leading causes of aseptic meningitis (the other usual suspects being echovirus and mumps virus). Coxsackieviruses share many characteristics with poliovirus. With control of poliovirus infections in much of the world, more attention has been focused on understanding the nonpolio enteroviruses such as coxsackievirus. (Sean P, Semler B L. Coxsackievirus B RNA replication: lessons from poliovirus. Curr Top Microbiol Immunol 2008; 323: 89-121).

Coxsackievirus B3 (CVB3) contains a positive RNA genome encoding a single open reading frame flanked by 5′ and 3′ untranslated regions (UTRs). CVB3 has a short lifecycle, which typically culminates in rapid cell death and release of progeny virus. Subsequent to virus attachment to receptors, viral RNA is released into the cell where it acts as a template for the translation of the virus polyprotein and replication of the virus genome.

B. MicroRNAs (miRNAs)

As noted above, the present invention provides miRNA-based approaches to modify the Enterovirus genome (e.g., a Poliovirus, Coxsackievirus or Echovirus genome) in order to further enhance its tumor-specificity. miRNAs are a class of endogenous small non-coding RNAs that are evolutionarily conserved and act as key regulators in a wide range of fundamental cellular functions by binding to the UTR of the targeted mRNAs. Subsequently, they promote either mRNA degradation or suppression of gene expression. Recent evidence suggests that miRNAs also play a key role in tumorigenesis. miRNAs are commonly observed to be downregulated in different cancer tissues. This unique feature can be exploited to develop miRNA-sensitive, tumor-specific oncolytic viruses. miRNA-145 (miR-145) and miR-143 have been identified as tumor-suppressive miRNAs and are significantly downregulated in lung cancer tissues.

Individual miRNAs and groups of miRNAs may be expressed exclusively or preferentially in certain tissue types. Exemplary miRNAs include miR-1, miR-7, miR-30c, miR-124, nniR-124*, miR-127, miR-128, miR-129, nniR-129*, miR-132, miR-135b, miR-136, miR-136*, miR-137, miR-139-5p, miR-143, miR-154, miR-184, miR-188, miR-204, miR-208, miR-216, miR-217, miR-299, miR-300-3p, miR-300-5p, miR-323, miR-329, miR-337, miR-335, miR-341, miR-369-3p, miR-369-5p, miR-375, miR-376a, nniR-376a*, miR-376b-3p, miR-376b-5p, miR-376c, miR-377, miR-379, nniR-379*, miR-382, nniR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*.

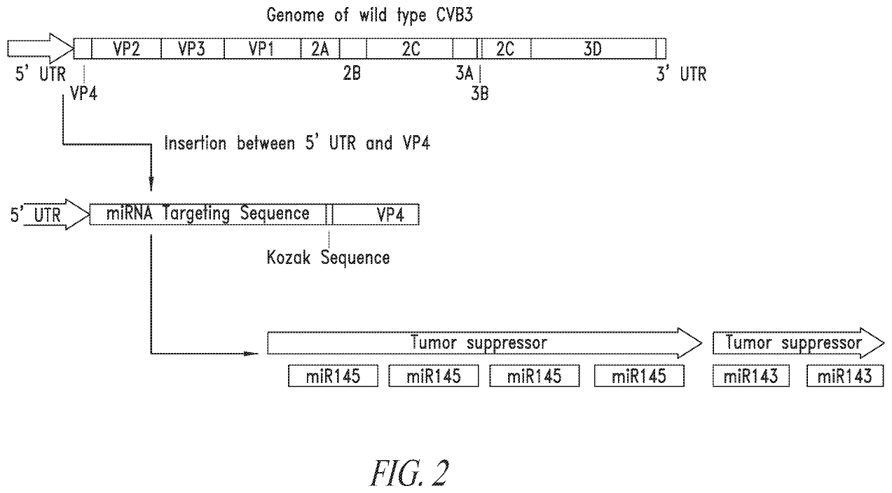

Within certain embodiments of the invention miRNA target sequences can be inserted in the 5′UTR or 3′UTR of the Coxsackievirus B3 genome. Within certain embodiments at least one, two, three, four, five, or six miRNA target sequences can be inserted in tandem. Within further embodiments there may be at least 10 target sequences can be inserted in tandem. Within other embodiments there are less than 10, 15, 20, 50, or 100 target sequences. An optimal number of target sequences can be determined by assaying expression levels of CSVB3. A low to nonexistent level of CSVB3 in normal cells is desired. The multiple miRNA target sequences may all bind the same miRNA or may bind different miRNAs. The target sequences may be in clusters (e.g., FIG. 2) in which for example, there are at least two target sequences in tandem that bind a first miRNA followed by at least two target sequences in tandem that bind a second miRNA and, optionally, followed by at least two target sequences that bind a third miRNA. Alternatively, the multiple miRNA target sequences that bind different miRNAs may be in no particular order. As well, there may be only a single copy of each miRNA target sequence. In some embodiments, there are 2-4 different miRNA targets. In other embodiments, there are 2-4 copies of each target sequence. In other embodiments, there are 2-4 different miRNA targets, and 2-4 copies of each of these target sequences in clusters. The miRNA target sequences may be inserted in any orientation or combination of orientations. See FIG. 2 for an exemplary construct.

The multiple miRNA target sequences may be adjacent without intervening nucleotides or have from 1 to about 25, or from 1 to about 20, or from 1 to about 15, or from 1 to about 10, or from 1 to about 5, or from 3 to about 10, or from 5 to about 10 intervening nucleotides. Intervening nucleotides may be chosen to have a similar G+C content as the 5′UTR and preferably do not contain a polyadenylation signal sequence.

C. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40-NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oncolytic virus to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 10¹¹ pfu. Within further embodiments, the dosage can range from about 10⁶ to about 10¹⁰ pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

E. Administration

In addition to the compositions described herein, various methods of using such compositions to treat or ameliorate disease (e.g., cancer) are provided, comprising the step of administering an effective dose or amount of a modified Coxsackievirus as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The OV (e.g., Coxsackievirus) as described herein may be given by a route that is e.g. intravenous, intratumor, or intraperitoneal. Within certain embodiments the oncolytic virus may be delivered by a cannula, by a catheter, or by direct injection. The site of administration may be intra-tumor or at a site distant from the tumor. The route of administration will often depend on the type of cancer being targeted. The OV (e.g., CSV) as described herein are particularly suitable for intravenous (IV) administration.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected. According to certain embodiments, treatment of a subject using the oncolytic virus described herein may be combined with additional types of therapy, such as chemotherapy using, e.g., a chemotherapeutic agent such as etoposide, ifosfamide, adriamycin, vincristine, doxycycline, and others.

OV (e.g., CSV) may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional

A therapeutically effective amount is preferably administered. This is an amount that is sufficient to show benefit to the subject. The actual amount administered and time-course of administration will depend at least in part on the nature of the cancer, the condition of the subject, site of delivery, and other factors.

Within yet other embodiments of the invention the oncolytic virus can be administered by a variety of methods, e.g., intratumorally, intraperitoneal, intravenously, or, after surgical resection of a tumor.

The following are additional exemplary embodiments of the present disclosure:

1. A replicating oncolytic virus vector comprising a modified Enterovirus genome, wherein the modified Enterovirus genome comprises one or more copies of one or more miRNA target sequences operably linked to an untranslated region (UTR) of the Enterovirus genome. Within a related embodiment, replicating oncolytic virus vectors ae provided comprising a modified Enterovirus genome, wherein the modified Enterovirus genome comprises a plurality of one or more miRNA target sequences operably linked to an untranslated region (UTR) of the Enterovirus genome. Within various embodiments the Enterovirus may be a Poliovirus, a Coxsackievirus, or an Echovirus.

2. The replicating oncolytic virus vector of embodiment 1, wherein said Enterovirus is a Coxsackievirus.

3. The replicating oncolytic virus vector of embodiment 2, wherein the Coxsackievirus is Coxsackievirus A or B.

4. The replicating oncolytic virus vector of any one of embodiments 1, 2 or 3, wherein the untranslated region (UTR) is a 5′ UTR. Within other embodiments the UTR is a 3′ UTR. Within yet further embodiments of the invention the one or more miRNA target sequences may be operably linked to a 3′ UTR and one or more miRNA target sequences may be operably linked to a 5′ UTR.

5. The replicating oncolytic virus vector of any one of embodiments 1, 2, 3 or 4, wherein the one or more copies of the one or more_miRNA target sequences comprises one or more copies of two or more different miRNA target sequences.

6. The replicating oncolytic virus vector of any one of embodiments 1, 2, 3, 4, or 5, wherein spacers of 2 to 50 base pairs (“bp”) in size are inserted between the one or more miRNA target sequences. Within various embodiments the spacers may be 2-10 bp, 10-20 bp in size, 20-30 bp in size, 30-40 bp in size, or 40-50 bp in size.

7. The replicating oncolytic virus vector of embodiment 1 wherein the one or more copies of the one or more miRNA target sequences recognize cardiac or pancreatic specific miRNAs. Representative examples of cardiac specific miRNAs include miR-1, miR133a/b, miR-208a/b and miR-499. Representative examples of pancreatic specific miRNAs include miR-7, miR-204, miR-216, miR-217, and miR-375.

8. The replicating oncolytic virus vector of any one of embodiments 1, 2, 3, 4, 5, 6, or 7, wherein the one or more different miRNA target sequences recognize an miRNA selected the group consisting of miR1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR216, miR217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873. By convention, the strand that is more frequently found to be the final product is referred to as miRNA and the rarer partner as miRNA*. Within various embodiments, the replicating oncolytic virus may contain one or more copies of positive strand miRNAs and/or one or more copies of negative strand miRNAs.

9. The replicating oncolytic virus vector of any one of embodiments 1, 2, 3, 4, 5, 6, 7, or 8, wherein the two or more (or plurality) of different miRNA target sequences comprise target sequences for miR1, miR133, miR216, miR145 and miR143.

10. The replicating oncolytic virus vector of embodiment 9, comprising two, three, four, five, or six copies of the target sequence for miR1, miR133, miR216, miR145 and miR143.

11. The replicating oncolytic virus vector of any one of embodiments 1, 2, 3, 4, 5, 6, 7, 8, 9, or 10, wherein one or more copies of the one or more miRNA target sequences is in a forward orientation and one or more copies of the one or more miRNA target sequences is in a reverse orientation.

12. The replicating oncolytic virus vector of any one of embodiments 1, 2, 3, 4, 5, 6, 7, 8, 9, 10, or 11, wherein the modified Enterovirus genome comprises at least one nucleic acid encoding a non-viral protein selected from the group consisting of immunostimulatory factors, antibodies (including for example bispecific antibodies), and checkpoint blocking peptides (also referred to as “checkpoint inhibitors” or “checkpoint modulators”), wherein the at least one nucleic acid is operably linked to a suitable tumor-specific regulatory region. Within various embodiments of the invention the bispecific antibody comprises a first antigen-binding domain which recognizes a tumor antigen, as well as a second antigen-binding domain which recognizes a cell surface molecule on an effector cell. Within other embodiments of the invention the checkpoint modulator is a peptide ligand, soluble domain of natural receptor, RNAi, antisense molecule or antibody. Within further embodiments of the invention the immune modulator at least partially antagonizes the activity of an inhibitory immune checkpoint(s), such as, for example, PD-1, PD-L1, PD-L2, LAG 3, Tim3, BTLA and /or CTLA4.

13. The replicating oncolytic virus vector of embodiment 12, wherein the non-viral protein is selected from the group consisting of IL12, IL15, IL15 receptor alpha subunit, OX40L, CD73, and a checkpoint inhibitor. Within further embodiments of the invention the above noted replicating oncolytic virus described in any of embodiments 1, 2, 3, 4, 5, 6, 7, 8, 9, 10, 11, or 12 retains its ability to infect and lyse tumor cells, but has reduced toxicity in vitro and/or in vivo as compared to an unmodified wild-type virus of the same strain (e.g., miR-modified coxsackievirus having greater than 5%, 10%, 25%, 50%, 75%, 80%, or 90% reduced toxicity in vitro and/or in vivo as compared to an unmodified wild-type coxsackievirus of the same strain). Within certain embodiments, the reduced toxicity is in cardiomyocytes, and/ or in normal pancreatic cells.

14. A method for lysing tumor cells, comprising providing an effective amount of a replicating oncolytic virus vector of any of embodiments 1, 2, 3, 4, 5, 6, 7, 8, 9, 10, 11, 12, or 13 to tumor cells. Tumor cells can be found, for example, in vivo within the cancers described herein,

15. The method of embodiment 14, wherein the tumor cells comprise lung cancer cells.

16. The method of embodiment 14, wherein the tumor cells comprise pancreatic cancer cells 17. Therapeutic composition comprising at least one replicating oncolytic virus vector of any of the above embodiments 1, 2, 3, 4, 5, 6, 7, 8, 9, 10, 11, 12, or, 13, and a pharmaceutically acceptable carrier.

18. A method for treating cancer in a subject suffering therefrom, comprising the step of administering a composition comprising a therapeutically effective amount of the composition of embodiment 17. Representative examples of cancers include those that are described herein. Particularly preferred cancers include lung cancers, pancreatic cancer, liver cancer and breast cancer.

19. The method of embodiment 18 wherein said cancer is selected from the group consisting of lung cancer, pancreatic cancer, liver cancer and breast cancer.

20. The method of embodiment 18, wherein the cancer is non-small-cell lung cancer (NSCLC) associated with KRAS mutations, small-cell lung cancer (SCLC) commonly linked to TP53 and Rb mutations, or pancreatic cancer.

21. The method of embodiment 18, 19, or 20 wherein the administration is intravenous (IV) administration, intraperitoneal (IP) administration, or intratumoral (IT) administration.

The following Examples are offered by way of illustration and not by way of limitation.

### EXAMPLES

### Example 1

### miR-145 and miR-143 are Downregulated in Lung Cancer Cells

This example describes an experiment to investigate the expression of miR-145 and miR-143 in lung and cardiomyocyte cells. miRNA levels were detected by reverse transcription reactions conducted with the following stem-loop primers: miR-145: CTCAACTGGTGTCGTGGAGTCGGCAATTCAGTTGAGAGGGATTC (SEQ ID NO: 1); miR-143: GTCGTATCCAGTGCTGGGTCCGAGTGATTCGCACTGGATACGACTGAGCTACA (SEQ ID NO: 2); and miR93:

CTCAACGGTGTCGTGGAGTCGGCAATTCAGTTGAGCTACCTGC (SEQ ID NO: 3). These primers were used to convert the cellular miR-145, miR-143 and miR-93 (internal control) into corresponding complementary DNA. Briefly, 1 μg total RNA from various lung cells and cardiomyocytes was mixed with 2 μl RT primer mix including 50 nM RT primer for each miRNA in 15 μl nuclease free water. Reactions were incubated at 65° C. for 5 min and then on ice for 2 min to form the stem loop. Then the mixture was used for the reverse transcription using iScript cDNA Synthesis kit (Biorad, 1708890) by mixing with 4 ul 5X iScript reaction mix and 1 μl reverse transcriptase at 25° C. for 5 min, 42° C. for 30min, and 85° C. 5nnin. The mixture was then diluted with 80 μl nuclease free water. For qPCR, three primer pairs were used to determine the relative expression level of miR-145 and miR-143: miR-145 (forward: CGGCGGGTCCAGTTTTCCCAGG (SEQ ID NO: 4); reverse: CTGGTGTCGTGGAGTCGGCAATTC (SEQ ID NO: 5)), miR-143 (forward: CCTGGCCTGAGATGAAGCAC (SEQ ID NO: 6); reverse: CAGTGCTGGGTCCGAGTGA (SEQ ID NO: 7)), miR-93 (forward: CGGCGGCAAAGTGCTGTTCGTG (SEQ ID NO: 8); reverse: CTGGTGTCGTGGAGTCGGCAATTC (SEQ ID NO: 9)). Briefly, 1 μl cDNA product was added into a 10 μl reaction mixture with primer concentration recommended by manufacturers using the Luna universal qPCR master mix (Neb, M3003). Reactions were cycled as follows: 95° C. for 1 min, 45 cycles of 95° C. for 10 s, 65° C. for 30 s, and then the melt curve was set in the ViiA 7 Real-Time PCR System (Applied Biosystems). Samples were run in triplicate and analyzed using a comparative CT (2—MCT) method with control samples and presented as relative quantitation (RQ).

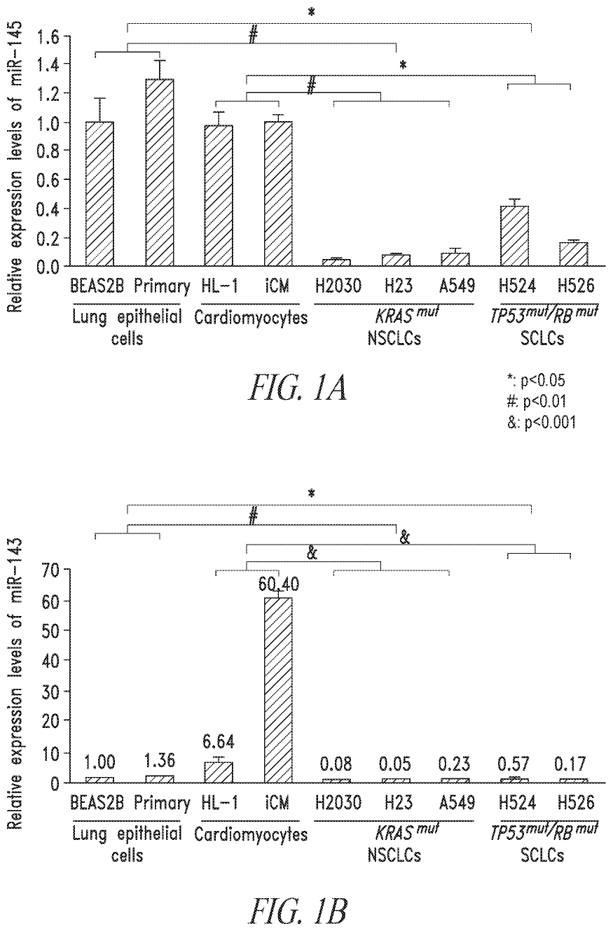

Results are presented in FIG. 1A (expression levels of miR-145) and FIG. 1B (expression levels of miR-143). These relative quantification results indicate that both miR-145 and miR-143 are significantly downregulated in lung adenocarcinoma cell lines with the KRAS mutation (H2030, H23, A549) and also in the SCLC cell lines with TP53 mutation (H524, H526) as compared to normal lung epithelial cells and cardiomyocytes.

### Example 2

### Construction of a miRNA-modified CVB3

To generate a recombinant CVB3 vector with decreased viral toxicity to normal tissues, a miRNA-engineered CVB3 was constructed as shown in FIG. 2. Four copies of the target sequence of miR-145 and 2 copies of the target sequence of miR-143 were inserted into a position between 5′UTR and start codon of VP4 to construct the miRNA-modified CVB3, denoted as miR-CVB3. The plasmid pCVB3/T7 containing the intact genome of CVB3 (Kandolf strain) was used as the backbone to generate miR-CVB3. Briefly, pCVB3/T7 was digested by Xbal to remove the BamHI sites while sparing the 5′UTR-VP4 region. The resulting plasmid was then mutagenized with a primer with the sequence, GTTGATACTTGAGCTCCCATTTTGCTGTATGGATCCTTTGCTGTATTCAACTTAACAATG SEQ ID NO: 10) harboring a BamHI site and a Kozak consensus sequence between 5′UTR and the start codon of VP4. The mutant backbone was further modified by inserting a BamHI-digested PCR product that includes 4-copy miR-145 target sequences and a Clal site amplified using a primer pair (forward: AATGGATCCTTAATTAACGAAGGGATTCCTGG (SEQ ID NO: 11); reverse: AATGGATCCTTAATTAAATCGATAGCGTCCAGTTTTC (SEQ ID NO: 12)) from the plasmid pCMV-ICP27-145T. The CVB3 genome in the resultant plasmid was then repaired by replacing the Bglll-Sall fragment with the corresponding fragment in pCVB3/T7 to construct pCVB3-miR145. Finally, the plasmid pCVB3-miR145-miR143 was generated by a Clal site insertion of an annealed oligo pair (forward: CGTGAGCTACAGTGCTTCATCTCACGATTGAGCTACAGTGCTTCATCTCATCTAGAAT (SEQ ID NO: 13); reverse: CGATTCTAGATGAGATGAAGCACTGTAGCTCAATCGTGAGATGAAGCACTGTAGCTCA (SEQ ID NO: 14)) including 2 copies of miR-143 target sequences. All restriction enzymes used here were from Thermo Fisher Scientific.

To produce miR-CVB3 and WT-CVB3 stock, viral genome was synthesized from pCVB3-miR145-miR143 and pCVB3/T7 linearized by Sall digestion, respectively, using HiScribe™ T7 Quick High Yield RNA Synthesis Kit (#E20505, New England Biolabs). Subsequently, viral RNA was transfected into HeLa cells and the supernatant was collected at ˜72 hours post-transfection when cytopathic effects were most prominent. The virus-containing supernatant was further propagated in HeLa cells until viral titers reached desirable levels for storage.

### Example 3

### Reduction of miR-CVB3 RNA Level, Viral Titers, and Cytotoxicity in Cardiomyocytes

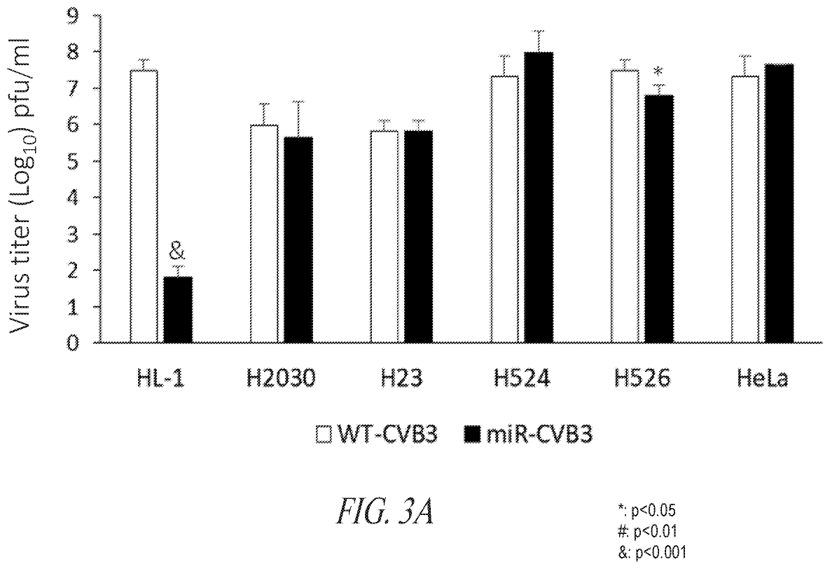

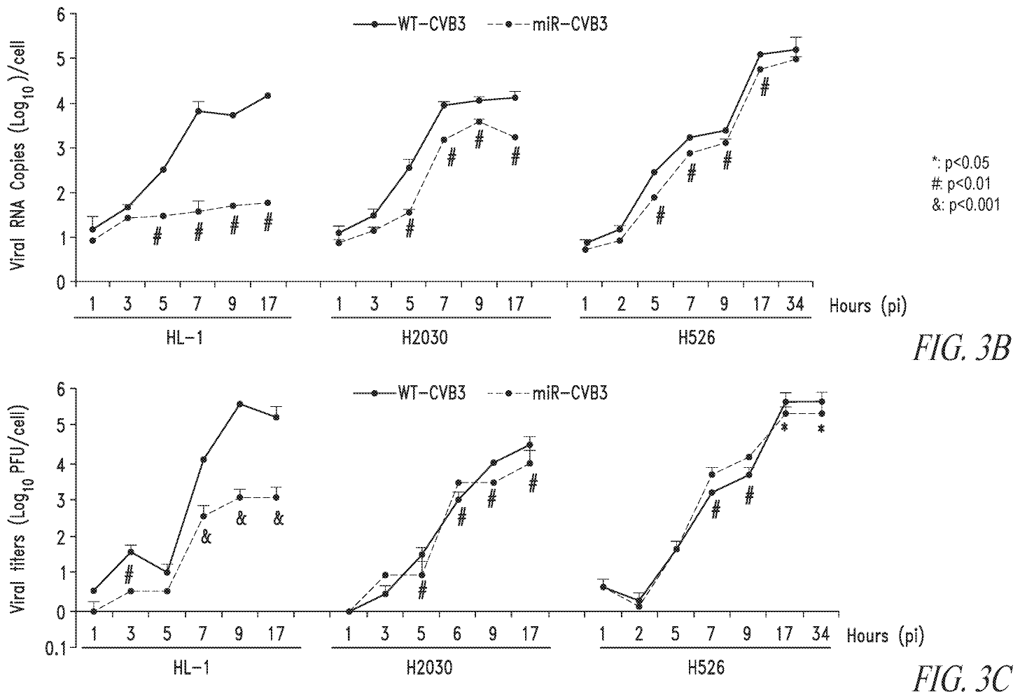

If the miRNA-modified CVB3 is susceptible to miRNA-dependent downregulation, it would be predicted that those cells with abundant miR-145 and miR-143 expression would selectively suppress the viral growth or replication of miR-CVB3 while WT-CVB3 growth would not be affected. To test this prediction, a panel of cell lines was treated with miR-CVB3 or WT-CVB3 at an MOI of 0.1 and a titration of viral particles in the supernatant 36 hrs post infection was performed. As shown in FIG. 3A, the titer of miR-CVB3 was significantly reduced compared to WT-CVB3 in HL-1 mouse cardiomyocytes, in which the miR-145 and miR-143 are highly expressed. In contrast, the titer of miR-CVB3 was not significantly reduced compared to WT-CVB3 in the NSCLC cell lines, SCLC cell lines and HeLa cells, in which the miRNA levels are downregulated.

The intracellular replication and one step growth curve of both viruses were also assessed and normalized to per cell levels by measuring the viral genome copy number (FIG. 3B) and viral titer (FIG. 3C) in cardiomyocyte HL-1 and lung cancer KRAS^{mut} H2030 and TP53^{mut} H526 cell lines. Significant differences were identified between the HL-1 cell line and the two lung cancer cell lines in the growth pattern of miR-CVB3 compared to WT-CVB3. In particular, replication of miR-CVB3 in HL-1 cells was strictly suppressed as reflected in the viral RNA copy levels and viral titer, while the WT-CVB3 genome and viral titer continued to amplify. In contrast, in both the H2030 and H526 cells, the replication of both viruses continued to amplify to around the same levels.

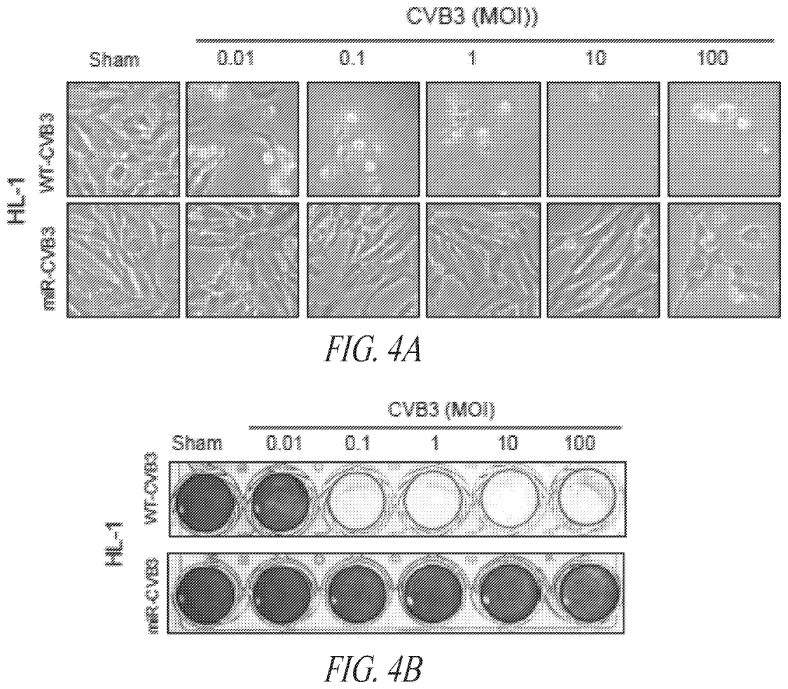

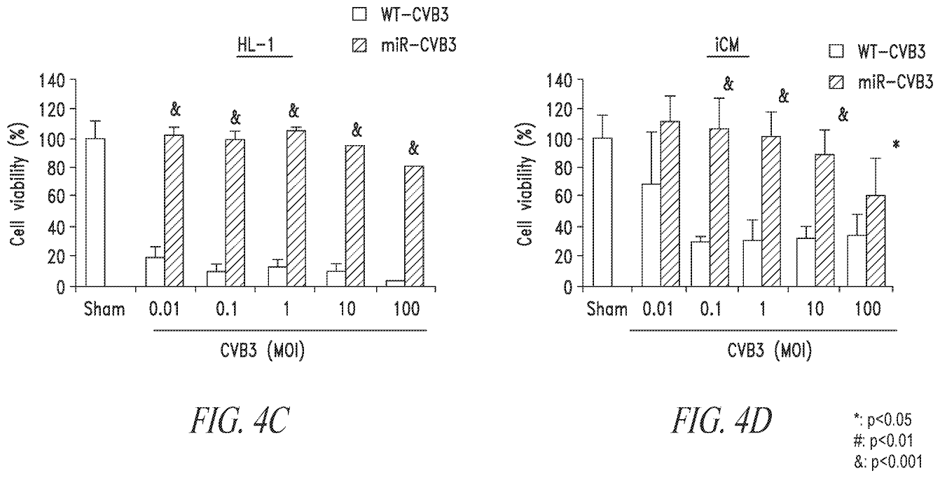

The toxicity of miR-CVB3 compared to that of the WT-CVB3 was tested in the mouse cardiomyocyte HL-1 cell line, that expresses relatively high levels of miR-145 and miR-143 as compared to lung cancer cells. As shown in FIGS. 4A-C, at initial virus infection MOIs of 0.01 to 100, significantly reduced toxicity of miR-CVB3 72 hrs post virus infection was observed. While WT-CVB3 causes HL-1 cell damage at an MOI of 0.01 to 0.1, it took a much higher dose of miR-CVB3 (MOI of 10 to 100) to induce clearly a cytopathic effect in HL-1 cells. The results of the morphology assay (FIG. 4A) were also reflected in the crystal violet staining test (FIG. 4B) that demonstrated empty wells in the WT-CVB3 treated cells as well as in the cell viability assay (FIG. 4C) by differential cell viability value. Since HL-1 cell lines are derived from tumor cells, human iPSC induced cardiomyocytes (iCM) were used to verify the cell viability assay, and the result was consistent to that tested in HL-1 cell lines (FIG. 4D).

### Example 4

### miR-CVB3 Retains the Ability to Lyse KRAS^{mut} Lung Cancer Cells

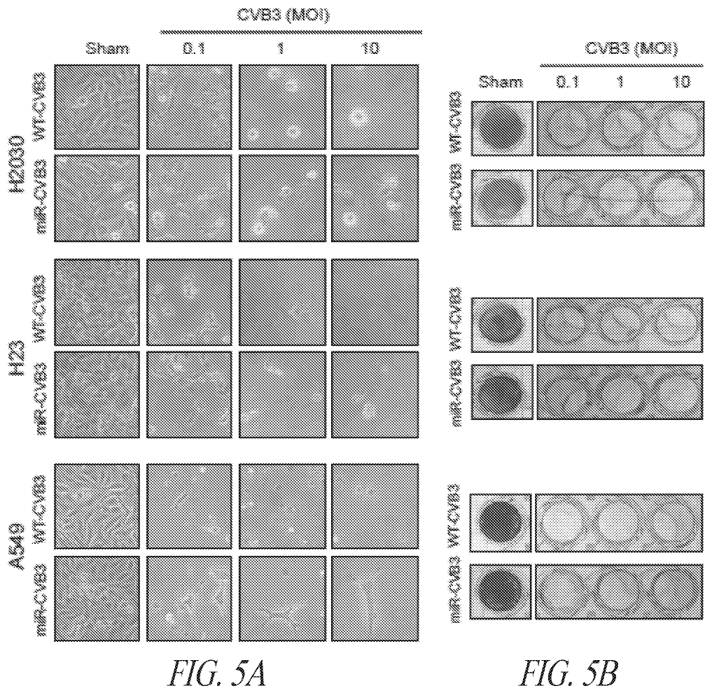

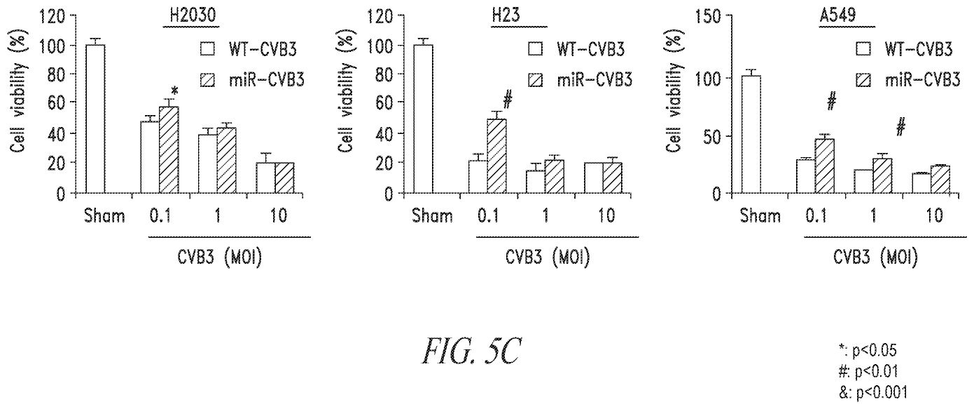

To determine whether the miRNA modification impairs the lytic ability of miR-CVB3 towards the KRAS^{mut} adenocarcinoma cells, three typical cell lines H2030 (G12C), H23 (G12C) and A549 (G12S), with mutant KRAS, were utilized for the evaluation. As shown in FIG. 5A and 5B, miR-CVB3 retains lytic ability against the three cell lines, with similar patterns of cell lysis as observed with WT-CVB3. Moreover, miR-CVB3 was observed to be weaker than WT-CVB3 in the ability to suppress the tumor cell viability (FIG. 5C) at a low MOI of 0.1. It can be speculated that artificial miRNA modification of CVB3 selectively boosts the innate immunity or antiviral ability against the miRNA-modified CVB3 in both normal cells and cancer cells, thereby accounting for the slightly reduced lytic ability of miR-CVB3 observed in cancer cells compared to WT-CVB3. Because the abundance of miR-145 and miR-143 in cardiomyocytes is substantially higher than that in cancer cells, it is predicted that cardiomyocytes should acquire greater anti-miR-CVB3 ability.

### Example 5

### Both WT- and miR-CVB3 Kill Small-Cell Lung Cancer (SCLC) Cells, Whereas Human Normal Lung

Epithelial Cells are Impermissive to both WT- and miR-CVB3.

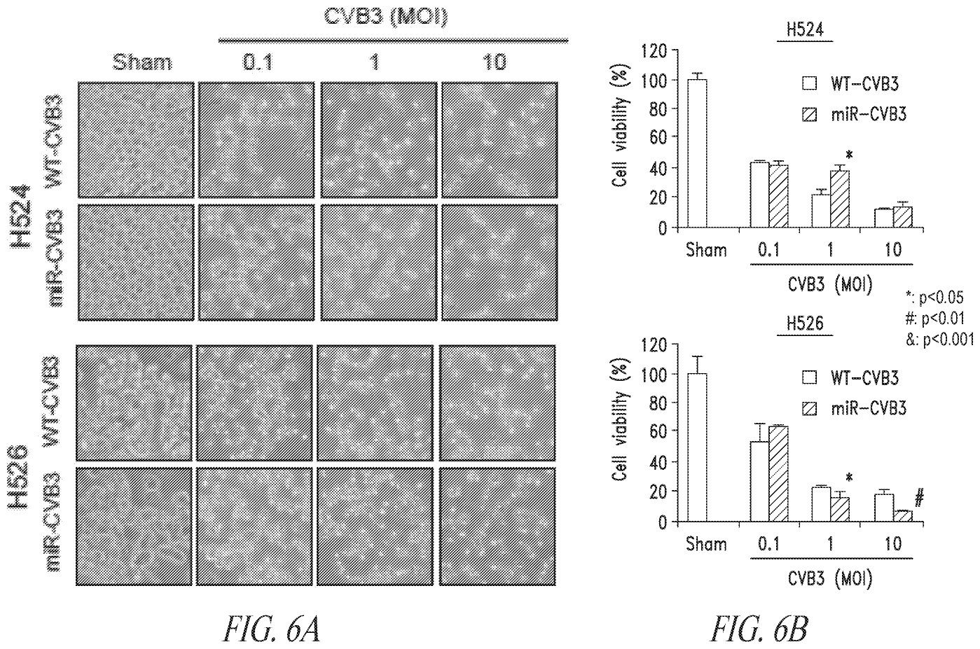

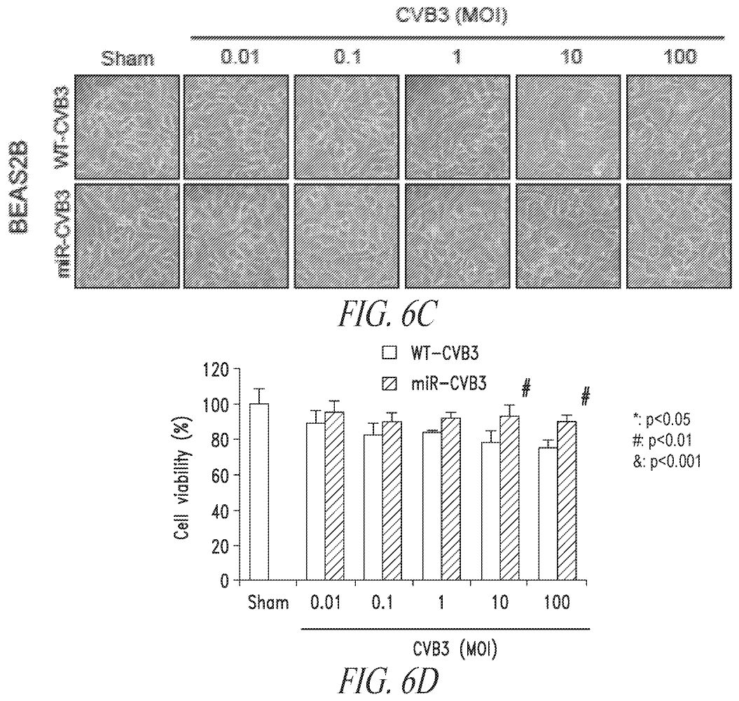

Compared to lung adenocarcinoma, SCLC has a closer correlation to smoking. H524 and H526, two suspension SCLC cell lines originally donated from two smoker patients, were seeded to begin to investigate 1) whether CVB3 has the potential to treat TP53^{mut}SCLC and 2) whether miR-CVB3 retains the equivalent ability to suppress the growth of SCLC cell lines. As shown in FIG. 6A, cells treated with WT-CVB3 and miR-CVB3 both exhibited aberrant cell morphology (e.g., shrinking size) as compared to the untreated control cells in the “sham” wells. This observation reflects the virus-induced cytotoxicity, which is also reflected in the cell viability assay results (FIG. 6B). BEAS2B, a normal lung epithelial cell line, was also tested in these experiments. As shown in FIG. 6C and 6D, BEAS2B is impermissive, or resistant, to both WT-CVB3 and miR-CVB3 even at an MOI of as high as 100.

### Example 6

### In Vivo Testing of miR-CVB3 in a Mouse Model

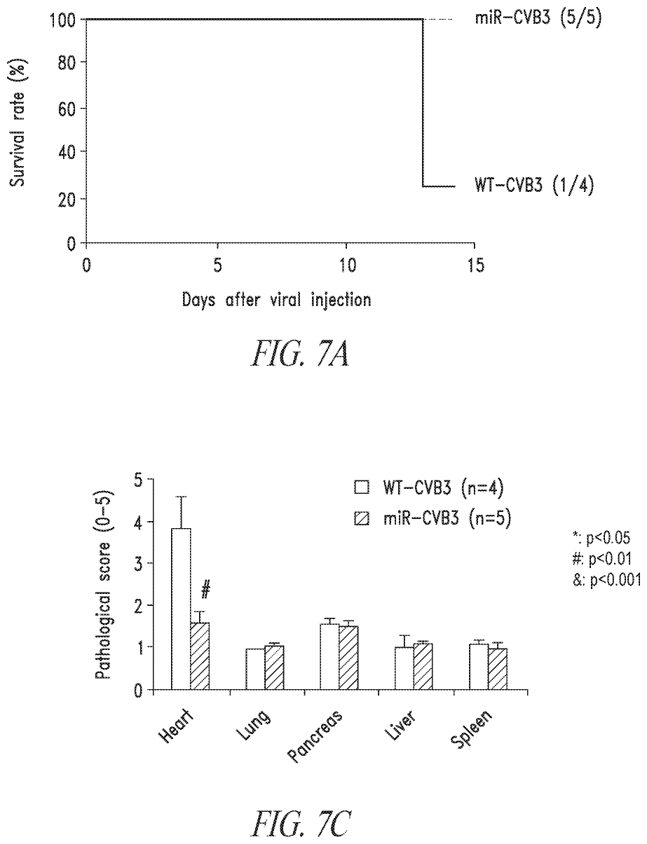

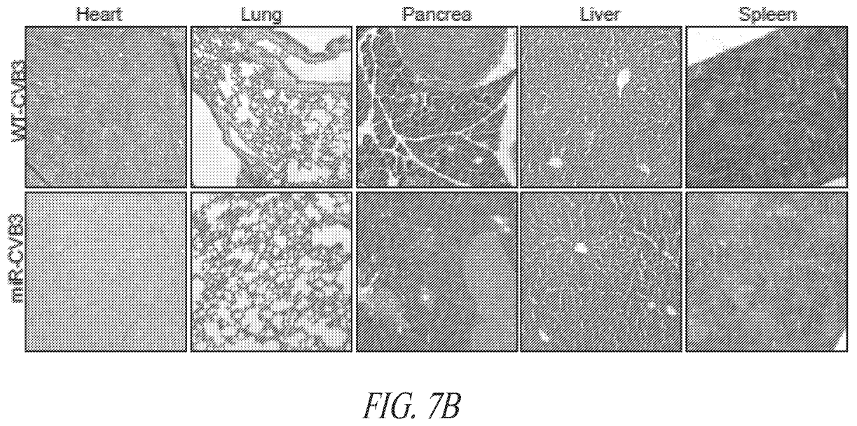

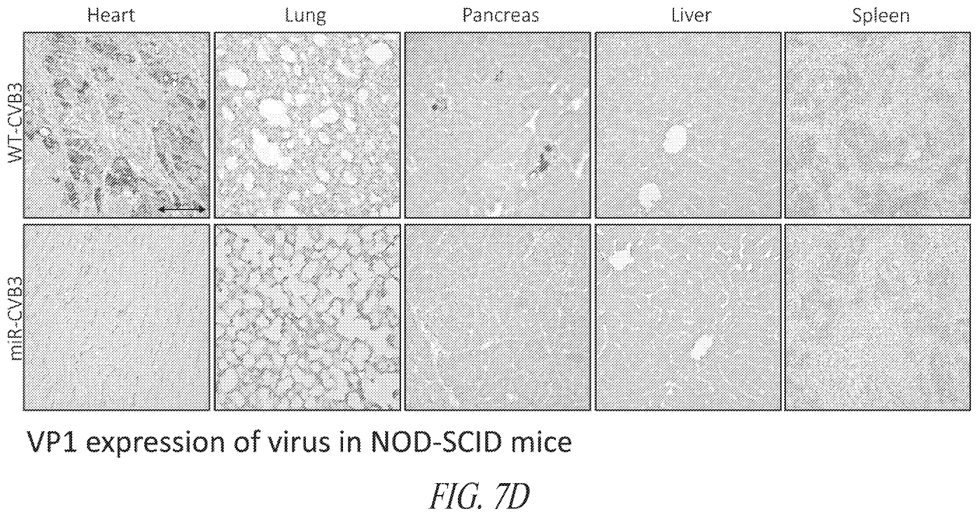

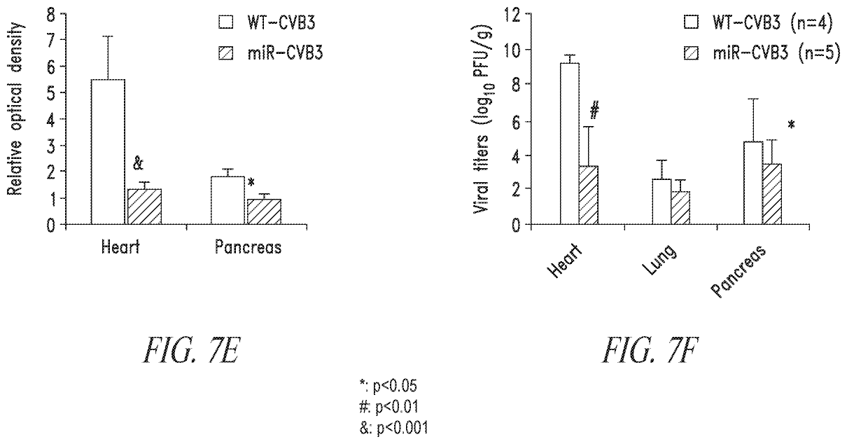

A safety test of miR-CVB3 compared to WT-CVB3 was done in immunocompromised NOD-SCID mice with an endpoint of day 14 post virus injection. 6-week-old mice were intraperitoneally administrated with WT-CVB3 (4 mice) or miR-CVB3 (5 mice) at a single dose of 1×10⁸ PFU. The treated mice were then monitored daily for change of body weight, appearance, behavior, and any signs of infection at the tumor cell injection site. The mice were kept in the cages until endpoint day 14, then euthanized. Heart, pancreas, lung, liver, kidney and spleen were collected for H&E and viral capsid protein VP1 staining, as well as viral plaque assay. As shown in FIG. 7A, NOD-SCID mice treated with WT-CVB3 showed severe toxicity and only one mouse survived (25% survival rate) throughout the time-course, while all mice treated with miR-CVB3 survived (100% survival rate) at the end of experiment. From the H&E staining slides shown in FIG. 7B, among the major organs, no significant pathological difference in lung, pancreas, liver and spleen between mice from two groups was observed, and the tissues appears to be normal based on the pathological score (FIG. 7C). Nonetheless, in the heart slides from mice treated with WT-CVB3, severe necrosis or tissue damage indicated by purple region was observed, which was not present in tissue slides from miR-CVB3 treated mice. In pathological score evaluation (FIG. 7C), high score tissue damage in the heart of WT-CVB3-treated, but not miR-CVB3 treated mice, was observed. It is speculated that the cardiotoxicity is almost abolished in miR-CVB3 treated mice. Viral quantitation by VP1 immunostaining (FIG. 7D and 7E) showed a significant reduction in viral protein VP1 expression (almost undetectable in the heart of miR-CVB3-treated mice) as compared to WT-CVB3 mice, indicating that decreased cardiac damage in miR-CVB3 mice is mainly due to reduced viral replication. It was also observed that VP1 expression is nearly undetectable in the pancreas of miR-CVB3-treated mice. As shown in FIG. 7F, live virus in heart, lung, and pancreas of mice from both groups was also measured, and the titer of miR-CVB3 is significantly reduced compared to that of WT-CVB3 in heart, but not that significant in pancreas and lung. There was no significant cardiovirulence by miR-CVB3; there was still live miR-CVB3 in heart, although at a low level.

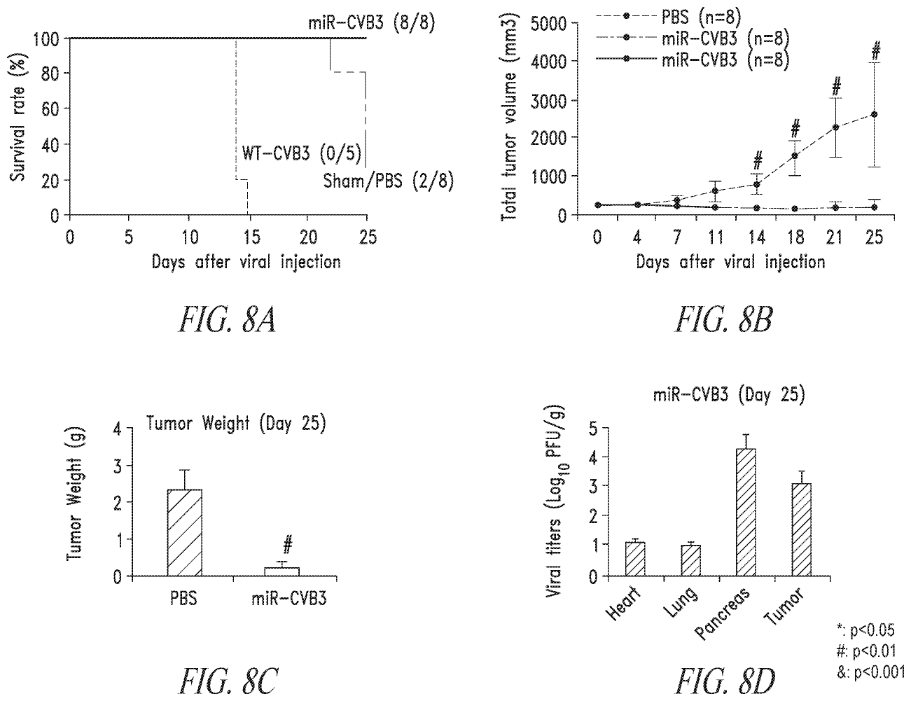

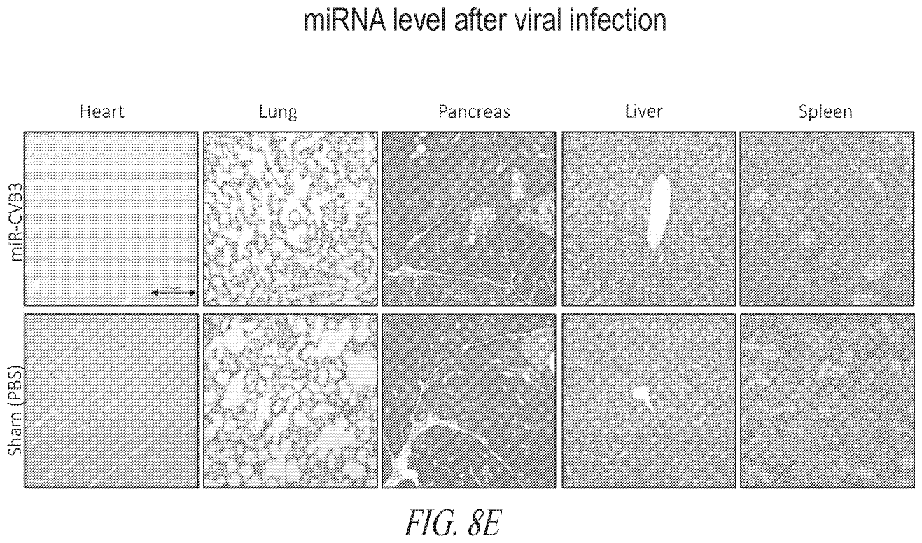

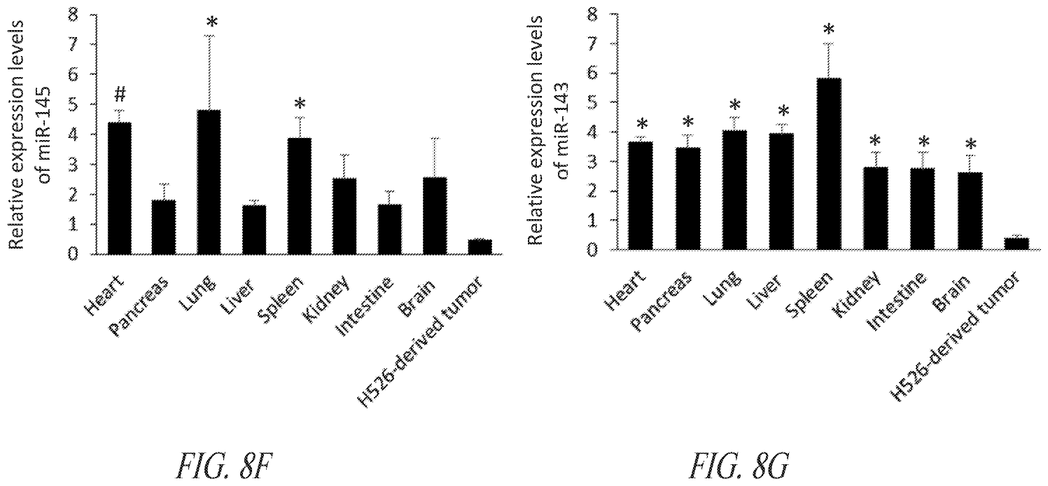

For efficacy testing of the miR-CVB3 oncolytic virus, the TP53^{mut} SCLC cell line H526 was used to establish a xenograft mouse model. Briefly, H526 cells (1×10⁷ cells) were injected subcutaneously into the left and right flank of 8-week-old male NOD-SCID mice. When tumors reached a size of around 100 mm³ (at around 10 days), mice were intraperitoneally injected with either PBS (8 mice), WT-CVB3 (5 mice,) or miR-CVB3 (8 mice) at a single dose (1×10⁸ PFU). Mice were monitored daily for general appearance, behavior, weight, and any signs of infection at the tumor cell injection site. Tumor size was measured twice per week and tumor volume was calculated as length×width×width/2. Mice were euthanized when they manifested severe symptoms related to CVB3 injection or until the endpoint day 25 unless the tumor diameter exceeded 2.0 cm. As the survival rate (FIG. 8A) indicates, until the endpoint day 25, all 8 miR-CVB3 treated mice looked normal, while 6 out of 8 PBS treated sham mice were euthanized due to oversized tumor; all 5 WT-CVB3 treated mice died or were euthanized due to morbidity at day 14. As the tumor growth curve (FIG. 8B) shows, tumors of PBS treated mice kept growing until the endpoint day 25, while tumors of both viruses treated mouse groups kept shrinking or the tumor growth was maintained at a low level. As expected, WT-CVB3 treated mice didn't survive after day 14. Implanted tumors and various organs were harvested on day 25. Tumor weight (mean +/−SD) (FIG. 8C) and viral titers (mean+/−SD) (FIG. 8D) were measured, and H&E staining (FIG. 8E) was conducted. #p<0.01 as compared to PBS sham controls. Expression levels of miR-145 (FIG. 8F) and miR-143 (FIG. 8G) were quantitated in the heart, pancreas, lung, liver, spleen kidney, intestine, brain, and H526-derived tumor of PBS-treated mice by qRT-PCR. The results are presented as mean+/−SD (n=3). An unpaired Student's t-test was performed for the comparison of the miRNA levels between different mouse tissues and H526 implanted tumors. *, p<0.05; #, p<0.01 compared to implanted tumors. These data indicate that the levels of miR-145 and miR-143 are significantly lower in implanted SCLC as compared to normal mouse tissues. In sum, these results indicate that miR-CVB3 retains the ability to inhibit tumor growth in a mouse xenograft model with substantially decreased cardiotoxicity.

### Example 7

### Construction of Additional miRNA-Modified CVB3s and In Vivo Testing in an Immunocompetent Mouse Model

Three additional recombinant miRNA-engineered CVB3 vectors were constructed by insertion of four copies of the target sequence of miR145 and two to four copies of the target sequences of miR-143, miR-1, miR-133, or miR-216 into the 5′ UTR of the CVB3 genome, denoted as miR-CVB3-A, miR-CVB3-B, and miR-CVB3-C (see FIG. 9A).

Male C57BL/6 mice aged ˜4 weeks were inoculated intraperitoneally with one dose of WT-CVB3 (n=3), miR-CVB3 (n=3), miR-CVB3-A (n=3), miR-CVB3-B (n=3), or miR-CVB3-C (n=3) at 1×10⁸ PFU or sham infected with PBS (n=3) for 14 days. Body weight reductions were observed in mice inoculated with WT-CVB3 and miR-CVB3, while mice inoculated with miR-CVB3-A, miR-CVB3-B, or miR-CVB3-C continued to gain body weight at a rate similar to that of mice inoculated with PBS control (see FIG. 9B). Intriguingly, mice treated with any of the miR-modified CVB3 vectors showed no signs of toxicity after 14 days, in sharp contrast to the mice treated with WT-CVB3, which reached a humane endpoint at 12 days and had to be euthanized (see FIG. 9C). Mouse organs were harvested for H&E staining and a pathological score was assigned to tissues isolated from the heart, lung, pancreas, liver, and spleen of each animal, with the highest pathological scores observed in the pancreas (see FIG. 9D).

### Example 8

### In Vitro Cardiotoxicity of Multiple miRNA-Regulated CVB3 Variants

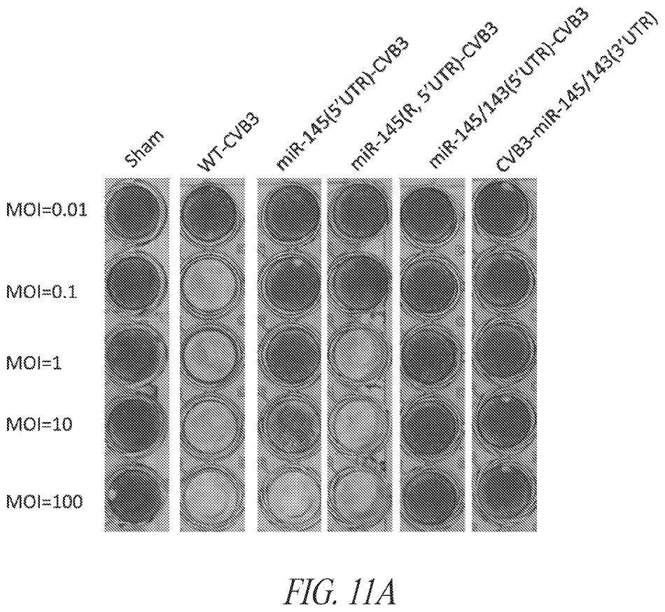

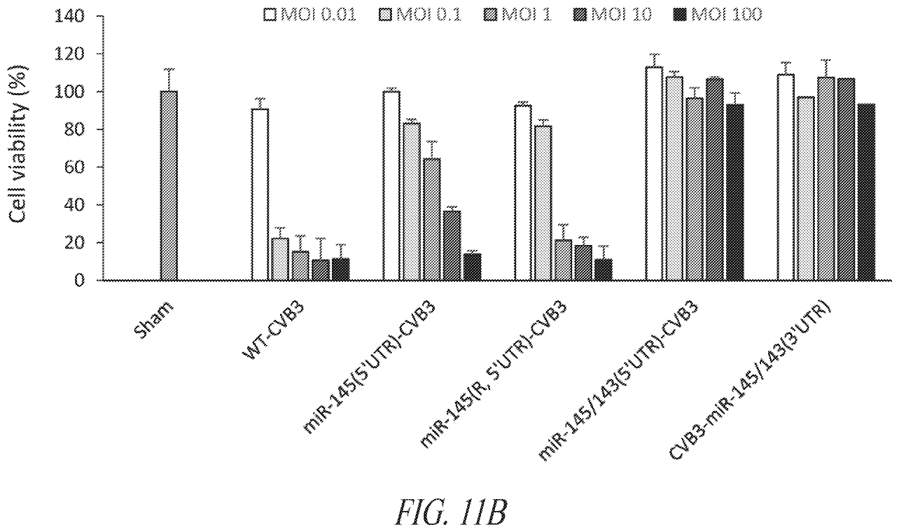

Several miRNA-modified CVB3s, in which four copies of miR-145 targeted sequences alone (either in its forward or reverse orientation) or in combination with two copies of miR-143 target sequences were inserted into the 5′ UTR or 3′ UTR of the CVB3 genome, were generated. Mouse HL-1 cardiomyocytes were sham-infected or inoculated with WT-CVB3 or various modified CVB3 at different MOIs as indicated for 72 hours. As shown in FIG. 11A, cytotoxicity was evaluated by crystal violet staining. As shown in FIG. 11B, cell viability was measured by the alamarBlue assay (mean +/−SD, n=3). miRNA-regulated CVB3s in which four copies of miR-145 and two copies of miR-143 target sequences were inserted into either the 5′ UTR or 3′ UTR of the CVB3 genome displayed the least cardiotoxicity in vitro. Based on these results, miR-145/143(5′ UTR)-CVB3 (denoted herein as miR-CVB3) was selected for further study.

### Example 9

### Modification of CVB3 by Additional miRNA-Targeting Sequences Further Reduces both WT-CVB3-Induced Cardio- and Pancreo-Toxicity in Immunocompetent Mice

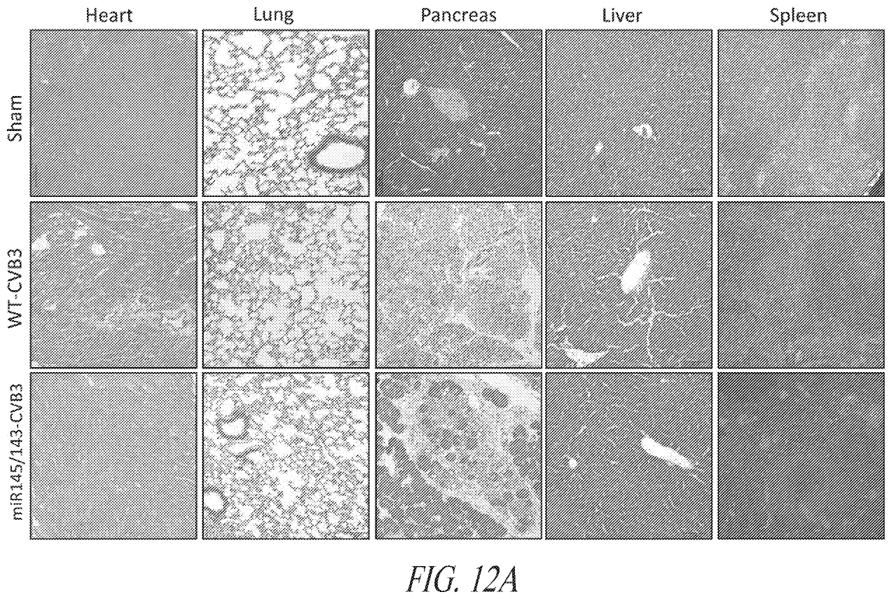

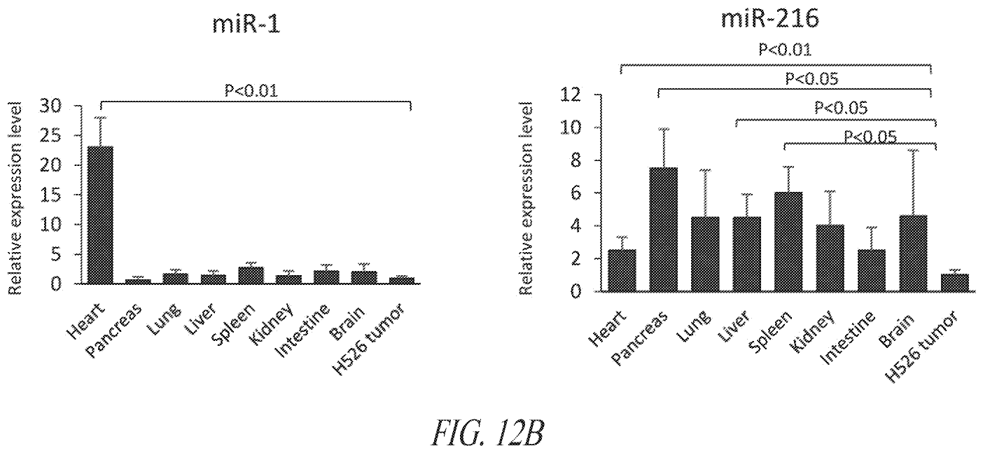

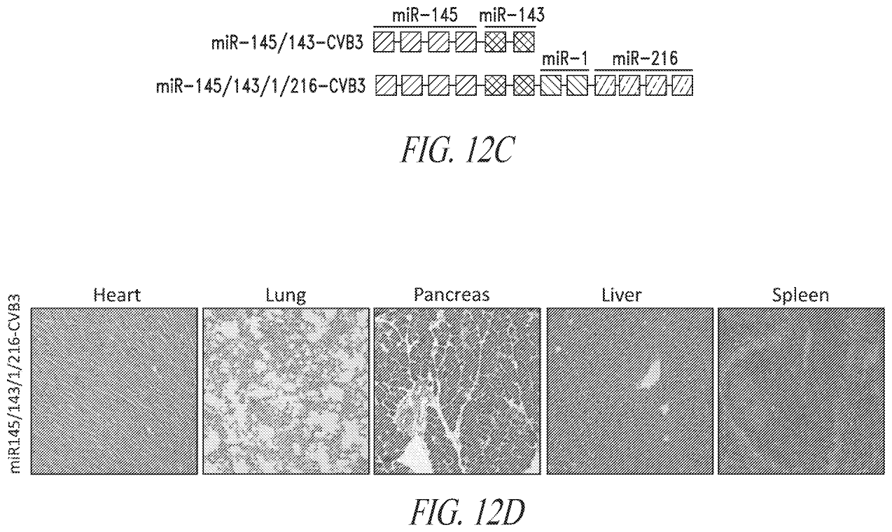

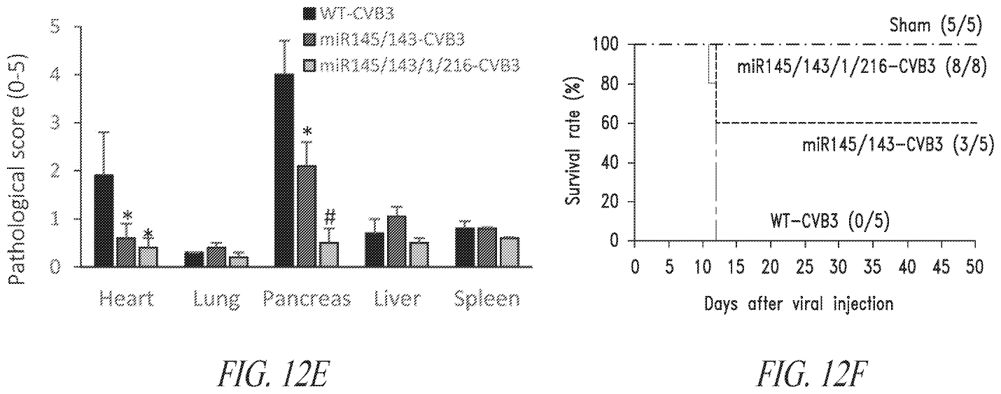

In this experiment, male C57B1/6 mice at the age of four weeks were inoculated intraperitoneally with one dose of PBS (sham, n=5), WT-CVB3 (1x10⁸ PFU, n=5), or miR145/143-CVB3 (1×10⁸ PFU, n=5). Mouse organs were harvested at day 12 for H&E staining (FIG. 12A). The levels of miR-1 and miR216 in different mouse organs and in human SCLC H526 cell-derived tumors were measured by RT-PCR (FIG. 12B). A novel miR-CVB3 was constructed as depicted in FIG. 12C. miR-145 and miR-143 are tumor suppressive, while miR-1 is enriched in muscle tissue and miR-216 is specifically expressed in the pancreas. These sequences show 100% homology between mice and humans. C57BL/6 mice were injected with miR145/143/1/216-CVB3 (n=3) as described above and organs were collected at day 14 post-treatment for H&E staining (FIG. 12D). Pathological scoring of the H&E staining depicted in FIGS. 12A and 12D is shown in FIG. 12E. *, p<0.05; #,p<0.01 as compared to the WT-CVB3 group. The Kaplan Meier plot of survival rate is shown in FIG. 12F.

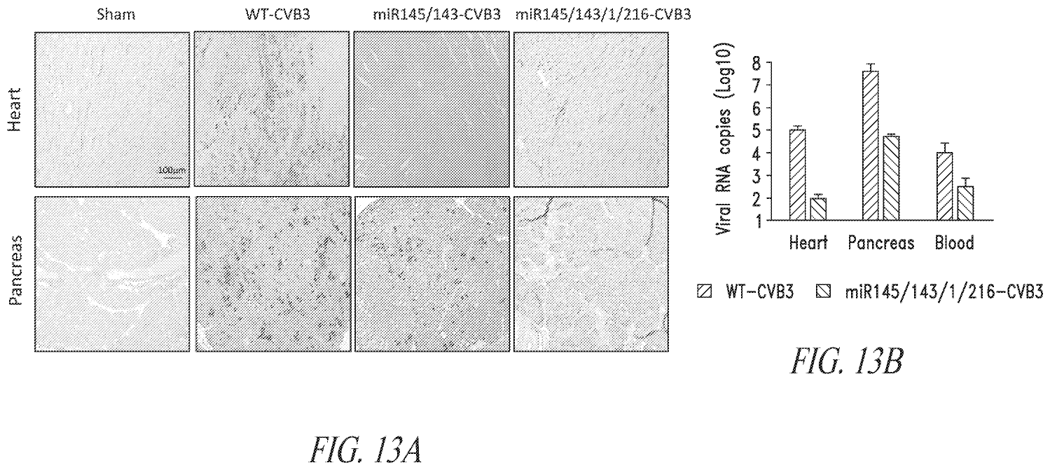

C57BL/6 mice were injected with various types of CVB3 as described above and organs were collected at day 13-14 post-treatment for IHC staining of viral protein VP1 (FIG. 13A) and RT-PCR analysis of viral RNA in different organs or blood (FIG. 13B).

### Example 10

### miRNA145/143/1/216-CVB3 Potently Kills SCLC Cells at a Comparable Level to WT-CVB3

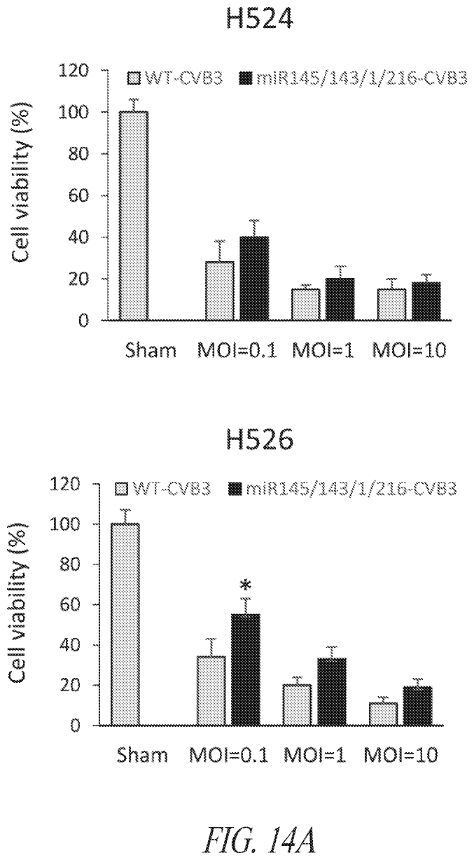

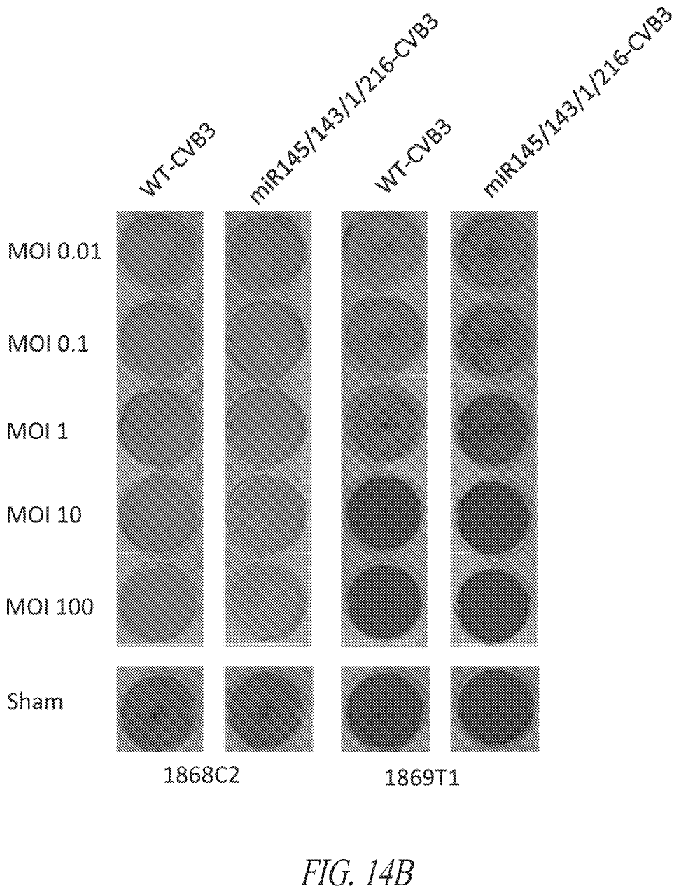

TP53^{mut}/Rb1^{mut} SCLC cells lines (H524 and H526) were sham-treated or infected with WT or miR145/143/1/216-CVB3 at different MOIs as indicated for 72 hours. Cell viability was assessed via the alamarBlue assay (mean +/−SD, n=3). *, p<0.05 compared to WT-CVB3 (FIG. 14A). Mouse TP53^{-/-}|Rb1^{-/-}|PTEN^{-/-} SCLC cells isolated from transgenic mice were treated as described above and cytotoxicity was assessed by crystal violet staining (FIG. 14B).

### Example 11

### miRNA-modified CVB3s Efficiently Kill a Variety of Mouse Tumor Cells

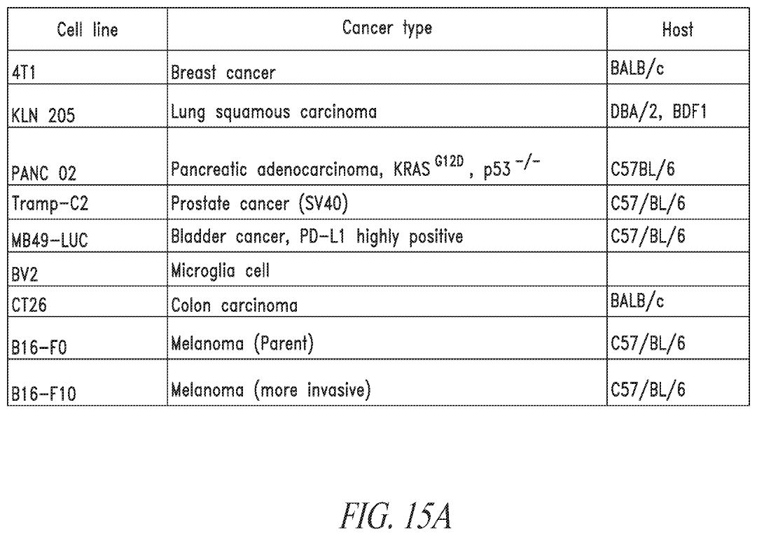

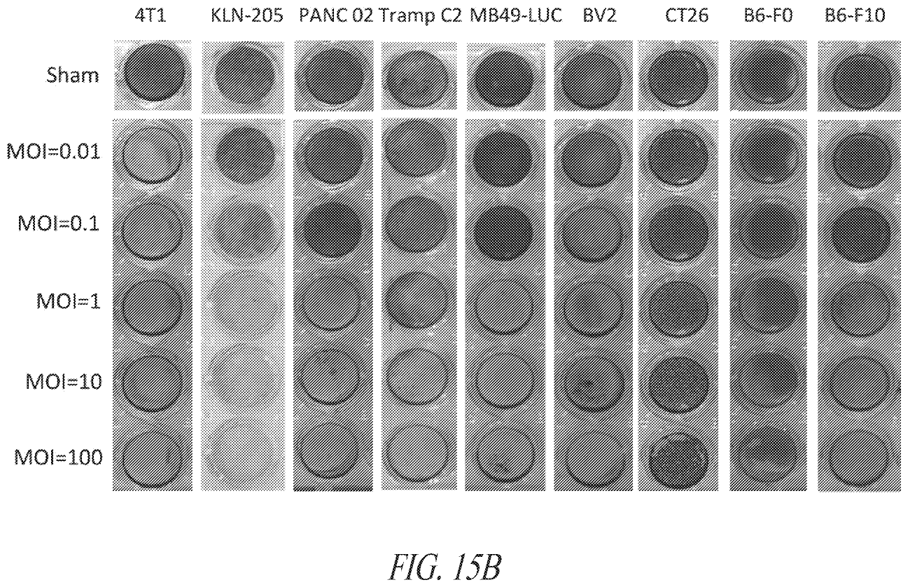

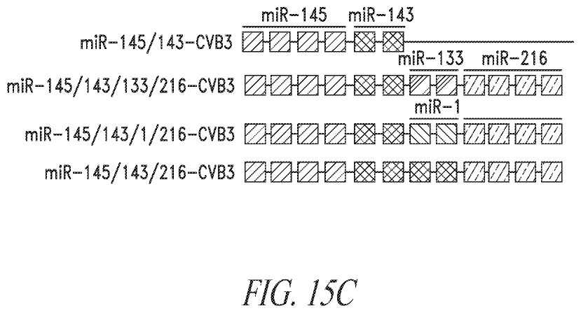

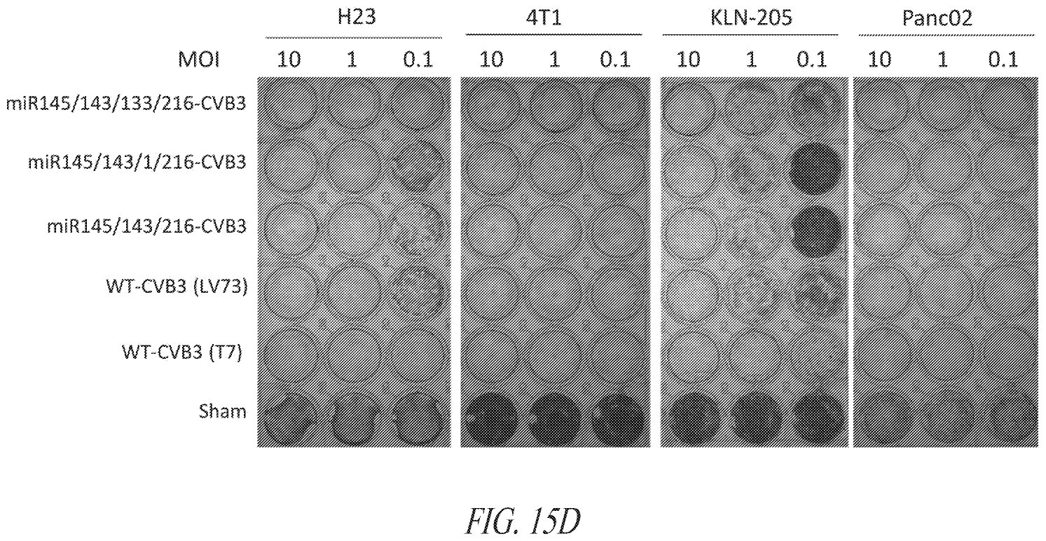

The various mouse tumor cell lines, cancer types, and host information used in this experiment are described in FIG. 15A. Cell lines were sham-infected or infected with miR145/143-CVB3 at different MOIs for 72 hours, followed by crystal blue staining (FIG. 15B). Three miR-CVB3 were constructed, as illustrated in FIG. 15C (miR-145/143-CVB3 (called “miR-CVB3” in FIG. 9A), miR-145/143/133/216-CVB3 (called “miR-CVB3-C” in FIG. 9A), miR-145/143/1/216-CVB3 (called “miR-CVB3-B” in FIG. 9A), and miR-145/143/216-CVB3 (called “miR-CVB3-A” in FIG. 9A)

Human KRAS^{mut} H23 cells and the various mouse tumor cell lines described above were sham-infected or infected with different miR-CVB3s at an MOI of 0.1, 1, or 10 for 72 hours, followed by crystal violet staining (FIG. 15D).

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A replicating oncolytic virus vector comprising a modified Enterovirus genome, wherein the modified Enterovirus genome comprises one or more copies of one or more miRNA target sequences operably linked to an untranslated region (UTR) of the Enterovirus genome.

2. The replicating oncolytic virus vector of claim 1, wherein said Enterovirus is a Coxsackievirus.

3. The replicating oncolytic virus vector of claim 2, wherein the Coxsackievirus is Coxsackievirus A or B.

4. The replicating oncolytic virus vector of claim 1, wherein the untranslated region (UTR) is a 5′ UTR or a 3′ UTR.

5. The replicating oncolytic virus vector of claim 1, wherein the one or more copies of the one or more miRNA target sequences comprises one or more copies of two or more different miRNA target sequences.

6. The replicating oncolytic virus vector of claim 1, wherein spacers of 2 to 50 base pairs in size are inserted between the one or more miRNA target sequences.

7. The replicating oncolytic virus vector of claim 1 wherein the one or more copies of the one or more miRNA target sequences recognize cardiac or pancreatic specific miRNAs.

8. The replicating oncolytic virus vector of claim 1, wherein the one or more different miRNA target sequences recognize an miRNA selected the group consisting of miR1, miR-7, miR-30c, miR-124, miR-124*, miR-127, miR-128, miR-129, miR-129*, miR-133, miR-135b, miR-136, miR-136*, miR-137, miR-139-5p, miR-143, miR-154, miR-184, miR-188, miR-204, miR-208, miR216, miR217, miR-299, miR-300-3p, miR-300-5p, miR-323, miR-329, miR-337, miR-335, miR-341, miR-369-3p, miR-369-5p, miR-375, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-499, miR-539, miR-541, miR-543*, miR-551b, miR-758, and miR-873.

9. The replicating oncolytic virus vector of claim 8, wherein the two or more different miRNA target sequences comprise target sequences for miR1, miR133, miR216, miR145 and miR143.

10. The replicating oncolytic virus vector of claim 9, comprising two, three, four, five, or six copies of the target sequence for miR1, miR133, miR216, miR145 and miR143.

11. The replicating oncolytic virus vector of claim 1, wherein one or more copies of the one or more miRNA target sequences is in a forward orientation and one or more copies of the one or more miRNA target sequences is in a reverse orientation.

12. The replicating oncolytic virus vector of claim 1, wherein the modified Enterovirus genome comprises at least one nucleic acid encoding a non-viral protein selected from the group consisting of immunostimulatory factors, antibodies, and checkpoint blocking peptides, wherein the at least one nucleic acid is operably linked to a suitable tumor-specific regulatory region.

13. The replicating oncolytic virus vector of claim 12, wherein the non-viral protein is selected from the group consisting of IL12, IL15, IL15 receptor alpha subunit, OX40L, CD73, and a checkpoint inhibitor.

14. A method for lysing tumor cells, comprising providing an effective amount of a first replicating oncolytic virus vector of claim 1 to tumor cells.

15. The method of claim 14, wherein the tumor cells comprise lung cancer cells.

16. The method of claim 14, wherein the tumor cells comprise pancreatic cancer cells, liver cancer cells, or breast cancer cells.

17. A therapeutic composition comprising at least one replicating oncolytic virus vector of any of the above claims and a pharmaceutically acceptable carrier.

18. A method for treating cancer in a subject suffering therefrom, comprising the step of administering a composition comprising a therapeutically effective amount of the composition of claim 17.

19. The method of claim 18 wherein said cancer is selected from the group consisting of lung cancer, pancreatic cancer, liver cancer and breast cancer.

20. The method of claim 18, wherein the cancer is non-small-cell lung cancer (NSCLC) associated with KRAS mutations, small-cell lung cancer (SCLC) commonly linked to TP53 and Rb mutations, or pancreatic cancer

21. The method of claim 18, wherein the administration is intravenous (IV) administration, intraperitoneal (IP) administration, or intratumoral (IT) administration.


SEQUENCE LISTING (14 sequences)

SEQ ID NO: 1 - DNA, 44 bp, Homo sapiens
ctcaactggtgtcgtggagtcggcaattcagttgagagggattc

SEQ ID NO: 2 - DNA, 53 bp, Homo sapiens
gtcgtatccagtgctgggtccgagtgattcgcactggatacgactgagctaca

SEQ ID NO: 3 - DNA, 43 bp, Homo sapiens
ctcaacggtgtcgtggagtcggcaattcagttgagctacctgc

SEQ ID NO: 4 - DNA, 22 bp, Homo sapiens
cggcgggtccagttttcccagg

SEQ ID NO: 5 - DNA, 24 bp, Homo sapiens
ctggtgtcgtggagtcggcaattc

SEQ ID NO: 6 - DNA, 20 bp, Homo sapiens
cctggcctgagatgaagcac

SEQ ID NO: 7 - DNA, 19 bp, Homo sapiens
cagtgctgggtccgagtga

SEQ ID NO: 8 - DNA, 22 bp, Homo sapiens
cggcggcaaagtgctgttcgtg

SEQ ID NO: 9 - DNA, 24 bp, Homo sapiens
ctggtgtcgtggagtcggcaattc

SEQ ID NO: 10 - DNA, 60 bp, Coxsackievirus B
gttgatacttgagctcccattttgctgtatggatcctttgctgtattcaacttaacaatg

SEQ ID NO: 11 - DNA, 32 bp, Homo sapiens
aatggatccttaattaacgaagggattcctgg

SEQ ID NO: 12 - DNA, 37 bp, Homo sapiens
aatggatccttaattaaatcgatagcgtccagttttc

SEQ ID NO: 13 - DNA, 58 bp, Homo sapiens
cgtgagctacagtgcttcatctcacgattgagctacagtgcttcatctcatctagaat

SEQ ID NO: 14 - DNA, 58 bp, Homo sapiens
cgattctagatgagatgaagcactgtagctcaatcgtgagatgaagcactgtagctca